(19) United States
(12) Patent Application Publication
(10) Pub. No.: US 2020/0005920 A1
(43) Pub. Date: Jan. 2, 2020

(54) Processing Pharmaceutical Prescriptions in Real Time Using a Clinical Analytical Message Data File

(71) Applicant: National Health Coalition, Inc., Fort Worth, TX (US)

(72) Inventors: Kenneth A. Hill, SR., Fort Worth, TX (US); Brad T. Crosslin, Keller, TX (US); Todd A. Crosslin, Aledo, TX (US); Clinton S. Ferguson, III, Arlington, TX (US)

(73) Assignee: National Health Coalition, Inc., Fort Worth, TX (US)

(21) Appl. No.: 16/567,742

(22) Filed: Sep. 11, 2019

Related U.S. Application Data
(63) Continuation-in-part of application No. 15/699,827, filed on Sep. 8, 2017.
(60) Provisional application No. 62/393,365, filed on Sep. 12, 2016.

(51) Int. Cl. G16H 20/10; G16H 10/60; G06Q 10/10; G06F 16/23; G06F 16/2455; G06N 20/00; G06F 17/18; G06F 9/54

(52) U.S. Cl. CPC G16H 20/10; G16H 10/60; G06Q 10/105; G06F 9/542; G06F 16/2455; G06N 20/00; G06F 17/18; G06F 16/2358

(57) ABSTRACT

A system and methods for automatically processing healthcare data associated with submission and fulfillment of pharmaceutical prescriptions by providers in real time, including claim processing, are enabled by a clinical services platform configured with a review processor and operable with a clinical analytical message (CAM) data file. The system includes the use of first and second databases respectively containing pharmaceutical data and standardized healthcare data. This data is extracted during processing by the system and translated into a common format for storage in a third electronic patient outcome record (EPOR) that is accessible, with full security and patient safety, to authorized providers and patients. Improved computer platforms configured to generate a portable, interoperable patient medical and pharmaceutical record, determine the compatibility of a prescription for a patient, and determine the prescription modification requirements for a patient is presented.

### CROSS REFERENCE TO RELATED APPLICATIONS

The present application is a continuation-in-part U.S. patent application Ser. No. 15/699,827 filed by the same inventors on Sep. 8, 2017, and entitled PROCESSING PHARMACEUTICAL PRESCRIPTIONS IN REAL TIME USING A CLINICAL ANALYTICAL MESSAGE DATA FILE, which claims the benefit of U.S. Provisional Application Ser. No. 62/393,365 filed by the same inventors on Sep. 12, 2016, and entitled HEALTHCARE DATA SYSTEM AND PROCESSES, the entire contents of each are incorporated herein by reference, in their entirety.

### BACKGROUND OF THE INVENTION

### 1. Field of the Invention

The present invention generally relates to management and integration of database information from multiple sources and more particularly to an electronic, patient-centric, portable, interoperable, interdisciplinary healthcare database system. The system includes healthcare data associated with prescription drugs, clinical services, physician and hospital and other healthcare provider services, and is designed for improving and reporting patient outcomes and providing processes for secure access by patients and their authorized healthcare providers.

### 2. Background of the Invention and Description of the Prior Art

It is widely known that the healthcare industry is highly complex. Massive amounts of data are involved in documenting every episode of care, and related transactions. And now the outcome reporting associated with every patient required by the Center for Medicare and Medicaid Services (“CMS”) adds to that complexity. Numerous healthcare providers participate in each episode of care. Healthcare service providers, including pharmacies, physicians, hospitals, laboratories, clinics, medical institutions, and regulatory agencies that develop clinical standards of care, historically have operated their own data systems, typically according to unique and different formats and processes.

An essential participant in healthcare services is another entity, the “payer” often an insurer, who pays for the services rendered and the claims made by the other healthcare providers. The insurance system includes the insurance companies that provide funds for payments to providers and to intermediaries that submit, adjudicate and facilitate payment transactions for services rendered to patients. In many instances it is difficult to timely access and use important data for patients' care and their outcomes. With new regulatory requirements that affect payments to healthcare providers and require reporting of patient measurable outcomes, further complexity and difficulties are assured if not adequately addressed.

The population of the United States, at the end of June, 2016, exceeded 324 million persons. Every person is a patient or potentially a patient of the healthcare system that requires medical attention and treatment, each with his or her unique healthcare needs and genetic profile, susceptibility to disease, and medical history. Many people contract an illness or are injured at some time during their lifetimes. The volume of patient healthcare data that accumulates during a patient's lifetime is enormous. Likewise, the data records of all of the healthcare providers who treat the patients, the data records of business entities including employers and insurance companies involved in payment transactions on behalf of the patients, and the records of governmental agencies involved in benefit programs and regulatory requirements associated with the healthcare of the patients adds to the volume of data.

The need for real time access and processing of this massive data is now essential to ensure that adequate healthcare services are delivered at the time of need and that the transactions and data associated with those services to patients are updated and completed with minimal delay. There is also then a need to promptly process reported measureable outcomes for treatment services, and ultimately payment.

An efficient healthcare system that maintains the health of persons active in the nation's economic enterprise is vital to its economic system. To accomplish the patient care required, it is essential that the data processing needs of all participating elements of the healthcare system be available to fulfill the demands of the healthcare system for timely, real-time access and handling of the massive amounts of data generated by the healthcare system. This is true for effective, cost-efficient healthcare services, and appropriate timely payment for same.

Handwritten record-keeping is clearly insufficient for these complex tasks. And the data processing systems operated by individual participants in the healthcare system have previously struggled to handle the tasks of keeping timely records, even with the aid of intermediaries that facilitate data interchange among participants. The ability to process the complex transactions involved in healthcare, and particularly for maintenance of patient records and for reimbursement of the fees and costs billed by providers—physicians, hospitals, laboratories and pharmacists—has accelerated faster than the capability to document and measure outcomes of the healthcare services (“Outcomes”) to the patients. Assurance of adherence to standards of care and improved efficiencies are now required by newly applicable regulations and must be realized with minimal delay.

Actions of the Federal Government in recent years have supplied some of the impetus toward upgrading the efficiency of the healthcare delivery system in the United States. Such actions include the Omnibus Budget Reconciliation Act of 1990 (OBRA '90), which requires drug utilization review procedures; an executive order by President George W. Bush in April, 2004, which set a ten year goal for “deployment of health information technology within 10 years;” and establishing the Health Information Technology for Economic and Clinical Health Act (HITECH Act), which sets forth “meaningful use of interoperable Electronic Health Records.” The HITECH Act, enacted as part of the American Recovery and Reinvestment Act of 2009, includes numerous categories of patient health records. And recently, the Center for Medicare and Medicaid Services (CMS) has established requirements for reporting patient outcomes of healthcare services delivered to patients in connection with approval for payments to providers.

What is needed is a comprehensive integration of the systems for obtaining and processing and utilizing healthcare patient data. To accomplish this task in real time, adaptable to the growth of the population, and to the needs of patients, providers and business organizations that serve the healthcare system, it is only possible by the use of highly developed computer systems that are configured in scalable ways for these essential tasks. There is no technology other than computers, networks, and systems for processing such a volume of data (“big data”) that will accommodate the need for data processing in the healthcare system. The need for continued innovation and improvement of the technology of the computing elements themselves, including particularly the software, is essential for the healthcare system to succeed in delivering the needed healthcare services and to meet the governmental regulatory requirements.

Innovation in healthcare delivery is a never-ending requirement. Well-developed software must be utilized to run efficiently to this end. Software and systems must be faster, with more efficient architectures and innovative ways to organize and structure the data processing environment.

### SUMMARY OF THE INVENTION

Accordingly, there is provided a healthcare data system providing storage, access and processing of prescription drug data, healthcare provider and clinical and laboratory services data, and patient outcome records in real time to be used by participating healthcare and service providers. The present invention solves the technological problem of multiple data transfers to large databases located across different networks. The present invention improves the performance of the system itself by reducing network congestion as multiple disparate databases do not need to be accessed for a single transaction, by allowing data to be accessible from a central location since patient data is consolidated into a single record. In contrast, a single database lacks the requisite patient information to provide meaningful information related to a prescription. Advantageously, the present invention enhances the patient data by combining data from other sources to create meaningful patient records for a service provider.

It is an object of the invention to provide a clinical services platform for gathering patient clinical and pharmaceutical data from separate sources in a single, common database configured for real time secure access by authorized providers and patients.

It is a further object of the invention to provide translation of the patient clinical and pharmaceutical data into a common format.

It is a further object of the invention to provide mechanisms in the clinical services platform for storing the translated patient clinical and pharmaceutical data into the single, common database in the common format to enable secure, real time access through a website portal into the system.

It is a further object of the invention to enable, through the clinical services platform real time, interoperable access to all relevant healthcare data in the one common database during prescription submission and fulfillment processing that are facilitated by the use of a clinical analytical message (CAM) data file.

It is a further object of the invention to provide updates in real time to the common database at every prescription transaction to ensure a complete and current record of all relevant healthcare data that is available to authorized healthcare providers.

It is a further object of the invention to provide secure, portable, interoperable access in real time to complete patient healthcare outcome data with enhanced safety.

These and other objects are provided by the following embodiments.

In one embodiment, there is disclosed a system for performing a review process of patient clinical data upon a submitted prescription from a healthcare provider in real time before it is received by a pharmacy for fulfillment, comprising a consolidated database that receives and stores patient clinical data from a plurality of sources in a patient clinical data record; a clinical analytical message data file (“CAM data file”) containing patient clinical data associated with the patient identified on the submitted prescription and the one or more pharmaceuticals identified on the submitted prescription; and a review processor operative on the CAM data file contents and with the database, the review processor including a suite of editing mechanisms for accessing the patient clinical data in the CAM data file, analyzing the patient clinical data in the context of the submitted prescription and information about the prescribed pharmaceuticals, and completing the review process.

In another aspect, the foregoing embodiment comprises a plurality of communication links for receiving the submitted prescription and transmitting (a) the submitted prescription to the pharmacy for fulfillment following the review process; (b) the CAM data file from one address in the system to another; and (c) the submitted prescription to the healthcare provider with revision suggestions and re-submitting the prescription to the pharmacy.

In another aspect, the CAM data file comprises a plurality of patient data fields; information resident in the patient data fields for completing drug utilization review (“DUR”), including at least: a plurality of patient clinical data retrieved from addressable locations in the database associated with the patient and the submitted prescription; and statements reporting results of analysis of the submitted prescription against patient clinical data retrieved from the database and analyzed by the software mechanism.

In other aspects, the CAM data file further comprises one or more of the following: information regarding required clinical education and services to be delivered to the patient with the dispensed prescription; a structure for cooperating with one or more editing mechanisms to perform analytical processing of stored patient clinical data to determine efficacy of the submitted electronic prescription; or a processing key associated with the one or more editing mechanisms.

In other aspects, the CAM data file may comprise an operative process including at least the following steps: receiving an electronic prescription submitted from a healthcare provider; entering patient and drug information into the CAM data file; subjecting the information entered into the CAM data file to executable code that analyzes the information with patient clinical data extracted from the database including at least one or more of information about potential drug interactions with other drugs and patient risk factors, including information selected from the group consisting of patient laboratory data, genomic data, immunizations, and allergies; and returning the CAM data file to the healthcare provider including suggestions or requirements for changes to the submitted electronic prescription before re-submitting the prescription.

In another aspect, the review processor may comprise a first process for automatically performing review of the submitted prescription and calling in sequence individual ones of the editing mechanisms to access, retrieve, and analyze the patient clinical data according to available information about the drug identified in the submitted prescription; a second process for controlling the plurality of communication links called during the pre-editing process; and a third process for selecting a pharmacy for fulfillment of an edited prescription based on pharmacy authorization data stored in the database corresponding to a prescribed drug identified in the edited prescription.

In another embodiment, in a system operating in a network including an electronic transaction hub (“switch”) and having a first database containing electronic patient outcome data (“EPO data”) coupled with a review processor that utilizes a clinical analytical message (“CAM”) data file, the invention embodies a process for performing a submission review of a pharmaceutical prescription submitted to a pharmacy (“a submitted prescription”), comprising the steps of logging into, by a healthcare practitioner, a healthcare website connected to the review processor in the network and adapted for entering the submitted prescription containing patient and medication information for the submission review; receiving via the healthcare website the submitted prescription into the review processor coupled to the healthcare website and to the first database containing patient clinical and pharmaceutical data from a plurality of sources; generating the CAM data file containing patient clinical data associated with the patient identified in the submitted prescription; obtaining EPO data from the first database that is relevant to the patient and the one or more pharmaceuticals identified in the submitted prescription; entering the EPO data into the CAM data file with the patient clinical data; attaching a key to the CAM data file to enable access by processing mechanisms; analyzing according to at least one submission review mechanism the patient clinical data and the prescription data in the CAM data file with the EPO Data to determine if the submitted prescription requires changes because of incompatibilities with the EPO data; notifying the healthcare practitioner of required changes and the submitted prescription must be re-submitted to the pharmacy with the required changes; or forwarding from the review processor the submitted prescription with the embedded CAM data file to the pharmacy through the electronic transaction hub for fulfillment by the pharmacy.

In another embodiment, in a system operating in a network including an electronic transaction hub (“switch”) and having a first database containing electronic patient outcome data (“EPO data”) coupled with a review processor that utilizes a clinical analytical message (“CAM”) data file, there is disclosed a process for performing a fulfillment review of a pharmaceutical prescription submitted to a pharmacy (“a submitted prescription”), comprising the steps of sending the prescription, accompanied by the CAM data file generated during a submission review, via the switch for a fulfillment review by the review processor, the CAM data file containing a payload including patient, clinical, and pharmaceutical information, to a pharmacy for dispensing; analyzing, according to at least one fulfillment review mechanism, the prescription to determine whether the prescription is for a specialty drug or is not for a specialty drug: if the prescription is not for a specialty drug, preparing the prescription for fulfillment; obtaining and delivering the prescription to the patient; if the prescription is for a specialty drug, copying required clinical and educational services information from the EPO data into the CAM data file of the patient; delivering required clinical and educational services information from the CAM data file to the patient; and generating via the CAM data file a message to one or more authorized recipients that the clinical and educational services information for the specialty drug were delivered, thereby authorizing for release a specified quantity of the specialty drug for dispensing to the patient; notifying a manufacturer of the specialty drug through a group purchasing organization that delivery of a specified quantity of the specialty drug is released; preparing and submitting, through the switch and a pharmacy benefit coalition to a payer, a first claim for delivering the clinical and educational services information and a second claim for dispensing the prescription to the patient; reconciling in the pharmacy benefit coalition payments against the first and second claims submitted to the payer; and crediting payments for the first and second claims to the pharmacy.

In another embodiment, a system for generating an electronic patient outcome record, the system including a server and a patient record database, can include: a server including computer-executable instructions that when executed cause the server to: identify a patient, in response to a first request from a user via a first computing device over an encrypted network, the first request including identifying information for the identified patient; receive pharmaceutical information entries for an identified patient from a plurality of pharmaceutical databases using the identifying information; reconcile, via a machine learning module, differences in the received pharmaceutical information entries by applying predetermined thresholds to the one or more fields of each of the plurality of pharmaceutical databases and generating reconciled pharmaceutical information entries for the identified patient using the pharmaceutical information entries satisfying the predetermined thresholds; generate a patient record in a patient record database, including a unique identifier and the reconciled pharmaceutical information entries for the identified patient; receive at least one of clinical, genomic, laboratory, disease, or standardized drug information for the identified patient from one or more data repositories; and update the patient record to include the received clinical, genomic, laboratory, disease, or standardized drug information for the identified patient. The system further comprising correlating, via the server, one or more fields of the identifying information from the first request with one or more fields of each of the plurality of pharmaceutical databases to identify whether they match. Wherein the pharmaceutical information entries include fields or parameters associated with information related to the patient. Wherein the first request is an electronic prescription. Wherein the electronic prescription can include fields or parameters related to specific attributes of the prescription, including a drug identifier, a dosage amount, or a dosage frequency. Wherein the patient record is a consolidated, reconciled database that is updated periodically, or whenever new data is available for a patient. Wherein the patient record is a consolidated, reconciled database that is updated whenever new data is available for the identified patient. Wherein the predetermined thresholds can be applied to the one or more fields of each of the identified patient entries, such that a certain tolerance can be attributed to the differences. Wherein the machine learning module identifies the identified patient entries satisfying the predetermined thresholds using a difference count or a difference percentage between the two identified patient entries being reconciled. Wherein if at least one of the received identified patient entries meets or exceeds at least one of the predetermined thresholds, the patient entry will be excluded from being reconciled with the other patient entries.

In another embodiment, a system for determining the compatibility of a prescription for a patient, the system including a server having a patient record database, and a machine learning module having a processor, can include: a server including computer-executable instructions that when executed cause the server to: identify a patient, in response to a first request from a user via a first computing device over an encrypted network, the first request including identifying information for the identified patient, receive pharmaceutical information entries for an identified patient from a plurality of pharmaceutical databases using the identifying information, reconcile, via a machine learning module, differences in the received pharmaceutical information entries by applying predetermined thresholds to the one or more fields of each of the plurality of pharmaceutical databases and generating reconciled pharmaceutical information entries for the identified patient using the pharmaceutical information entries satisfying the predetermined thresholds, generate a patient record in a patient record database, including a unique identifier and the reconciled pharmaceutical information entries for the identified patient, receive at least one of clinical, genomic, laboratory, disease, or standardized drug information for the identified patient from one or more data repositories, and update the patient record to include the received clinical, genomic, laboratory, disease, or standardized drug information for the identified patient; and a machine learning module having a processor configured to: conduct a patient analysis to establish a patient profile corresponding to one or more health concerns based at least in part on the identified patient's pharmaceutical, clinical, genomic, laboratory, disease, or standardized drug information, receive a prescription for the identified patient, the prescription having one or more prescription parameters including a drug identifier, a dosage amount, or a dosage frequency, correlate the one or more prescription parameters with at least one of the pharmaceutical, clinical, genomic, laboratory, disease, or standardized drug information for the identified patient to determine an incompatibility, and generate and transmit an alert to the user indicating whether the prescription is compatible with the identified patient. Further comprising correlating, via the server, one or more fields of the identifying information from the first request with one or more fields of each of the plurality of pharmaceutical databases to identify whether they match. Wherein the identified patient's pharmaceutical, clinical, genomic, laboratory, disease, or standardized drug information includes parameters or fields related to specific attributes of the patient's information. Wherein the patient profile includes a listing of health concerns, a health rating, or one of a predetermined number of health concern levels indicating severity of the health concern. Wherein the thresholds have minimum or maximum values for parameters having a scale, magnitude, or degree. Wherein the system further comprises a feedback loop configured to modify the thresholds using the patient record. Wherein the machine learning module modifies the thresholds by calculating a difference, standard deviation, average, or interpolation of the patient record. Wherein the patient analysis to establish the patient profile generates a health concern level for a patient. Wherein the machine learning module generate one or more health concern definitions to establish the thresholds for the prescription parameters. Wherein the alert is a clinical analytical message (CAM) data file.

In another embodiment, a system for generating prescription modification requirements for a patient, the system including a server having a machine learning module and a patient record database, can include: a server comprising computer-executable instructions that when executed cause the server to: receive, by the server over an encrypted network, a prescription for a patient, the prescription having prescription parameters including a drug identifier, a dosage amount, and a dosage frequency; retrieve, by the server, a record from a database having patient information and standardized data, including pharmaceutical, clinical, genomic, laboratory, disease, or drug information; generate, by the server, one or more health concern definitions including one or more health concern parameters related to the patient information and standardized data; determine, by the machine learning module, whether one or more of the prescription parameters satisfy one or more parameters of the health concern definitions; if the one or more of the prescription parameters satisfy one or more parameters of the health concern definitions, determine, by the server, one or more alternative prescription parameters and return, by the server, a modification requirement alert including the one or more alternative prescription parameters; and if the one or more of the prescription parameters do not satisfy one or more parameters of the health concern definitions, return, by the server, an acknowledgement. Wherein the health concern definitions can be satisfied by determining whether differences between the prescription parameters and the health concern parameters meet, exceed, or fall below the thresholds or health concern definitions. Further comprising correlating one or more of the prescription parameters with one or more parameters of the health concern definitions for the identified patient to determine any differences. Further comprising determining whether the differences fall below the predetermined thresholds, match the predetermined thresholds, or exceed the predetermined thresholds. Wherein the health concern definitions relate to health concerns or potential issues for the identified patient based on health related information. Wherein the alternative prescription parameter modifies the dosage using the patient information and standardized data that falls within acceptable thresholds. Further comprising receiving drug incompatibility information from the standardized drug database. Wherein if the drug identifier prescription parameter satisfies one or more parameters of the health concern definitions, a binary indication of the presence of the drug incompatibility generates a modification requirement alert. Further comprising receiving the patient's condition information from the clinical database and querying the standardized drug database to find similar drugs that can treat the same condition, but that do not have an incompatibility. Wherein the alert is a clinical analytical message (CAM) data file.

### BRIEF DESCRIPTION OF THE DRAWINGS

FIG. 1 illustrates a system network diagram depicting major functional components of one embodiment of the present invention;

FIG. 2 illustrates a functional block diagram of front end processing of a specialty prescription drug according to one embodiment of the invention;

FIG. 3 illustrates a functional block diagram of back end processing of a specialty prescription drug according to the embodiment of FIG. 2;

FIG. 4A illustrates a flow chart diagram of a first portion of the front end processing performed by the system depicted in FIG. 2;

FIG. 4B illustrates a flow chart diagram of a second portion of the front end processing performed by the system depicted in FIG. 2;

FIG. 5A illustrates a flow chart diagram of a first portion of the back end processing performed by the system depicted in FIG. 3;

FIG. 5B illustrates a flow chart diagram of a second portion of the back end processing performed by the system depicted in FIG. 3;

FIG. 6 illustrates one example of a data bit structure that may be used in one embodiment of the invention;

FIG. 7 illustrates a system network diagram depicting major functional components of another embodiment of the present invention;

FIG. 8 illustrates a flow chart diagram for generating a portable, interoperable patient medical and pharmaceutical record performed by the system depicted in FIG. 7;

FIG. 9 illustrates a flow chart diagram for determining the compatibility of a prescription for a patient performed by the system depicted in FIG. 7; and

FIG. 10 illustrates a flow chart diagram for determining prescription modification requirements for a patient performed by the system depicted in FIG. 7.

### DETAILED DESCRIPTION OF THE INVENTION

The advancement in technology described herein meets the need for real time processing, updating, and accessing of the massive amounts of data involved in the delivery of healthcare. It is a system best characterized as a new, real time “Patient-Centric, Portable, Interoperable, Interdisciplinary, Electronic Patient Outcome Record” that is produced by and functions cooperatively within a new arrangement of principal components in a healthcare data system that resides in an electronic healthcare network. It is a network that links participants in the system—patients and authorized healthcare providers, data resources and processing elements, payers, pharmaceutical manufacturers, and pharmacies—in the delivery of healthcare services to patients through designated portals with each other and with the comprehensive database system. A chief feature of the system is that it ensures safe, secure, real time access to an Electronic Patient Outcome Record (“EPOR”) by authorized healthcare providers including their patients through portals to the databases within the network. The data resources including the EPOR are updated in real time with every healthcare transaction to ensure the availability of timely and accurate information to the participating healthcare providers and their patients. The system is structured around a network of three database components and a clinical services platform that includes specially developed software. The three database components include a structured healthcare database called electronic patient outcome data (“EPO data”) [1^{st} component]; an electronic pharmacy record (“ePR”) database [2^{nd} component]; and an electronic patient outcome record (“EPOR”) database [3^{rd} component] that is populated by data from the EPO data and ePR databases. The specially developed software that provides operational control is embodied in a new clinical services platform. Additional software is embodied in a pharmaceutical delivery system that interfaces with a National Health Information Network (“NHIN”) to supply data and services to the EPO database and process claims for payment for services.

The system described herein integrates the data storage and processing facilities of the healthcare industry in a way that achieves its real time operability through the use of specialized software systems—generally residing in cloud storage to be described—and accessible throughout the system from strategic locations. The computer systems that provide access to this network are necessarily distributed throughout the healthcare system. This real time interoperability is organized, in part, around the processes and transactions involving the prescribing of ethical drugs by physicians for treatment, delivery of the drugs through the pharmaceutical system, and processing the payments to healthcare providers and pharmacies for healthcare services rendered, all according to standardized rules and procedures established by industry practice and in compliance with federal regulatory requirements for documenting and reporting measurable patient outcomes.

The new structures of the system build on some existing elements of the healthcare system, primarily those that are configured for processing and documenting the delivery of healthcare services and products by providers. These providers include physicians, hospitals, laboratories and pharmacies, which provide healthcare services and products and seek reimbursement from payers for billed fees and expenses for those services and products. The new structure integrates the existing elements with the new electronic healthcare system components described herein. The system provides for an electronic patient outcome record (EPOR) database, bidirectional communication portals into the system and the ePR and EPO data databases, and the clinical services platform for standardized access. The resulting combination provides the aforementioned “Patient-Centric, Portable, Interoperable, Interdisciplinary, Electronic Patient Outcome Record”. This system operates to manage the processing of prescription drugs, from a full and complete automatic submission review followed by automatic fulfillment review including delivery of essential clinical and educational services, dispensing of the pharmaceutical to the patient, and processing of the claims for payment or reimbursement. The system also documents data associated with the delivery of healthcare services including patient outcomes as needed for payment as required by the Center for Medicare & Medicaid Services (“CMS”).

The features and benefits of the system could not be achieved in real time without the software specifically designed to ensure that this processing provides continually updated data and access to it throughout every patient—provider encounter and transaction associated with each episode of healthcare provided to each patient. A further key component of the system to be described is an innovative data file that travels with and is involved in the transactions processed by the system. As is well known in the art, a data file is a component of a data record comprised of a sequence of fields for the entry of data. A database is a storage repository for data records. This “Clinical Analytical Message” or “CAM” data file is an integral part of the system that enables accurate and complete, real time processing of the submission and fulfillment transactions involving specialty prescription drugs. Moreover, these features and benefits would not be possible without the combination of the high speed capabilities of computer processing, direct access to the various databases in the system through the designated portals within the system, and the reliance on redundant data structures and extensive use of cloud storage. Thus, the system to be described relies on a combination of data processing mechanisms, both hardware and software, to configure the system to achieve the data processing results in real time—i.e., at nearly the same time as the system is accessed to enter a prescription submission or check on the status of a patient record, for example.

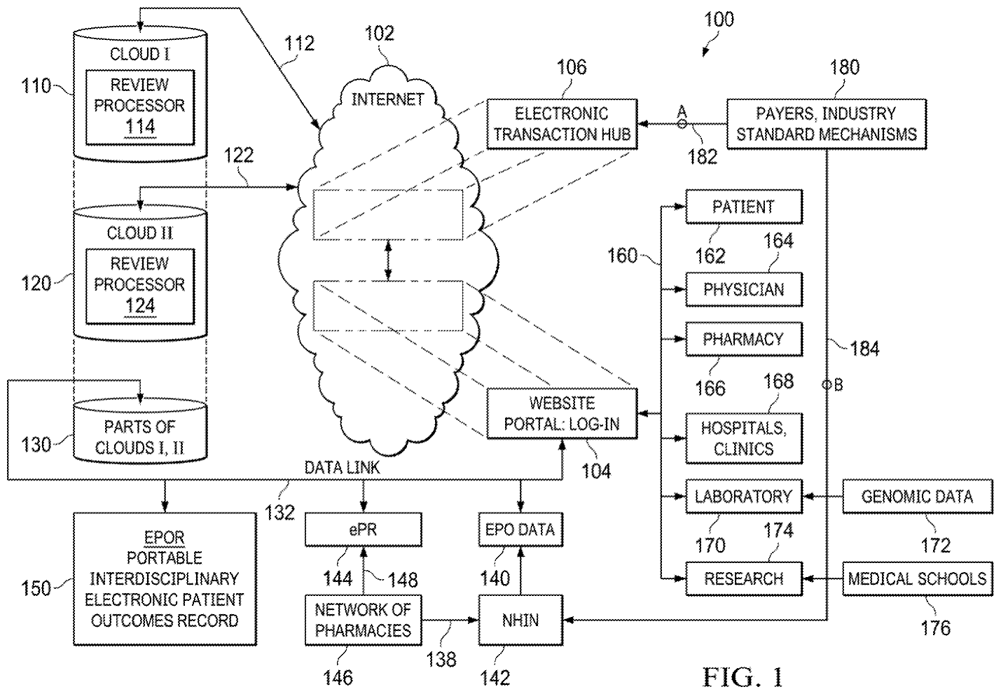

The present invention thus provides a new system architecture for processing healthcare data that is depicted in FIGS. 1, 2, and 3. The invention comprises a combination of data processing mechanisms arranged and configured for efficiently processing both prescription submissions and fulfillment, as well as healthcare data updates and secure patient outcome data accessibility in real time to all authorized healthcare providers and patients. The technological improvements embodied in the present invention include configuring the following three innovations: (1) an enhanced database organization system; (2) an enhanced (automated) prescription submission and fulfillment processing system provided by a new clinical services platform; and (3) a data file format for facilitating data movement and processing in both systems (1) and (2).

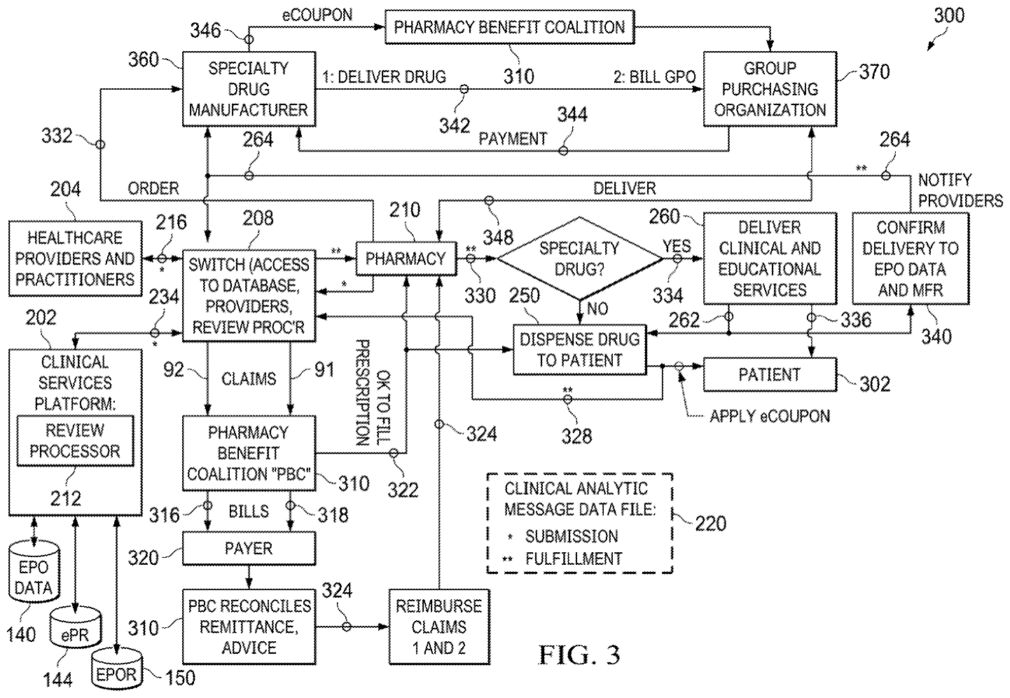

The system and processes to be described herein, while applicable to all types of prescriptions, will be described in the context of prescribing, fulfilling, documenting and reporting a specialty drug prescription issued by a physician and processing the associated interactions and transactions among participants and providers in the system, including the processing of payments by payers to providers of products and services to patients. A principal feature of the process for prescribing the specialty drugs is that they are limited to special order status from the manufacturer of the specialty drug. Typically this transaction is handled through a Group Purchasing organization or GPO. The GPO forwards the order from the pharmacy to the manufacturer. The manufacturer then fills the order for the specialty drug from inventory, per the prescription, along with an invoice to the GPO for the costs of the drug. Upon payment, the prescription is released for delivery to the dispensing pharmacy. Upon receipt of the specialty drug, the pharmacy arranges for delivery of the required clinical and educational services information to the patient, and for delivery of the specialty drug to the patient or to the person or entity authorized to administer the drug. Subsequently the pharmacy submits separate requests for payment to the patient's insurance carrier of the claim for delivery of the clinical and educational information and the claim for dispensing the specialty drug to the patient. The payment portion of these transactions is preferably handled by a Pharmacy Benefit Coalition (“PBC”), also called a Pharmacy Benefit System or Office herein, that will be described with FIG. 3.

The Pharmacy Benefit Coalition is an entity or office formed by the inventor of the present system as an enhanced alternative to the industry's pharmacy benefit manager (or “PBM”) system. The PBC acts as a coordinating body for pharmaceutical benefits available from payers associated with the patient—usually an insurance company or employer by contract or benefit plan or Federal or State benefits such as Medicare and Medicaid. The PBC is devised to efficiently manage the payer benefits to the patient by clearing e-prescriptions (“eRx”) for fulfillment and facilitating the transactions associated with fulfilling the e-prescription.

During the processing of payments for a specialty drug, an eCoupon, processed by the Pharmacy Benefit Coalition, accompanies the specialty drug en route to the GPO for delivery to the pharmacy. In the illustrated embodiment, the GPO also remits payment for the specialty drug to the manufacturer.

### Overview of Key System Concepts

It will be helpful to understanding the description that follows by briefly reviewing several key concepts embodied in the present invention. The concepts are: (1) real time access by all authorized participants in the healthcare system to complete clinical and pharmaceutical data for each patient, that is updated in an electronic patient outcome record (EPOR) at each healthcare transaction; (2) complete pharmaceutical files for each patient includes, in addition to the descriptive information about the drug, interaction data with both other drugs and foods, and drug-specific laboratory and genomic information; and (3) an essential functional principle of the system is the capability to provide the clinical analytical message (“CAM”) data files by which key information is conveyed and delivered as needed during the process of prescribing, reviewing, distributing, and dispensing of specialty and non-specialty prescription drugs, for and to the patient, and managing and recording measureable outcomes related to treatment for the patient. An additional concept (4) of a patient specific algorithm that functions as a secure doorway of the patient's file is also discussed.

The real time accessibility of clinical and drug data means that the files of the EPO data database and the electronic pharmacy record (ePR) database are accessible and updated during read/write transactions by healthcare providers authorized to access the data. The EPOR, populated from the EPO data and ePR databases, is a consolidated database that may also be updated at each transaction to help ensure that accurate data is available for the next healthcare provider that accesses the record so that healthcare provider actions and decisions are informed by the data. This concept helps assure patient safety by maintaining timely updates to the patient record, a primary objective of the healthcare system disclosed herein. Regarding specialty drugs, the delivery of those drugs to a patient may not proceed or be completed unless a current, accurate, and comprehensive patient data record is available and satisfies the clinical requirements for such fulfillment. Real time updates to the data files also facilitates ongoing healthcare research evaluation of required clinical data.

The completeness of pharmaceutical and clinical files, created for each patient, is essential to enable prescribing specialty drugs that are compatible with the metabolic and clinical profile of the individual patient to help ensure (a) that the most effective drug is prescribed; (b) that a drug or dosage that can be adequately and safely metabolized will be prescribed; (c) that side effects are minimized, including those that could be potentially dangerous; and (d) that potentially dangerous interactions with other drugs or foods are minimized. The pharmaceutical drug files as configured in the system transcends the need for formularies—tiers and lists of different categories of branded and generic drugs used by payers to limit drug dispensing to the cheapest drug rather than the most appropriate drug.

In another application of the pharmaceutical and clinical data files, chain retailers offering both grocery and pharmacy products are excellent candidates for utilizing the benefits of these comprehensive pharmaceutical files that form an integral part of the present healthcare system disclosed herein. The availability of such files, when incorporated into a retail environment that also provides, for example, food products purchased by the patient, enables interaction cross checks to be performed easily from data resident in the same database system used by the retail business. This analysis capability helps to avoid dispensing drugs incompatible with the patient's diet and clinical profile, and with other substances that may be consumed by the patient.

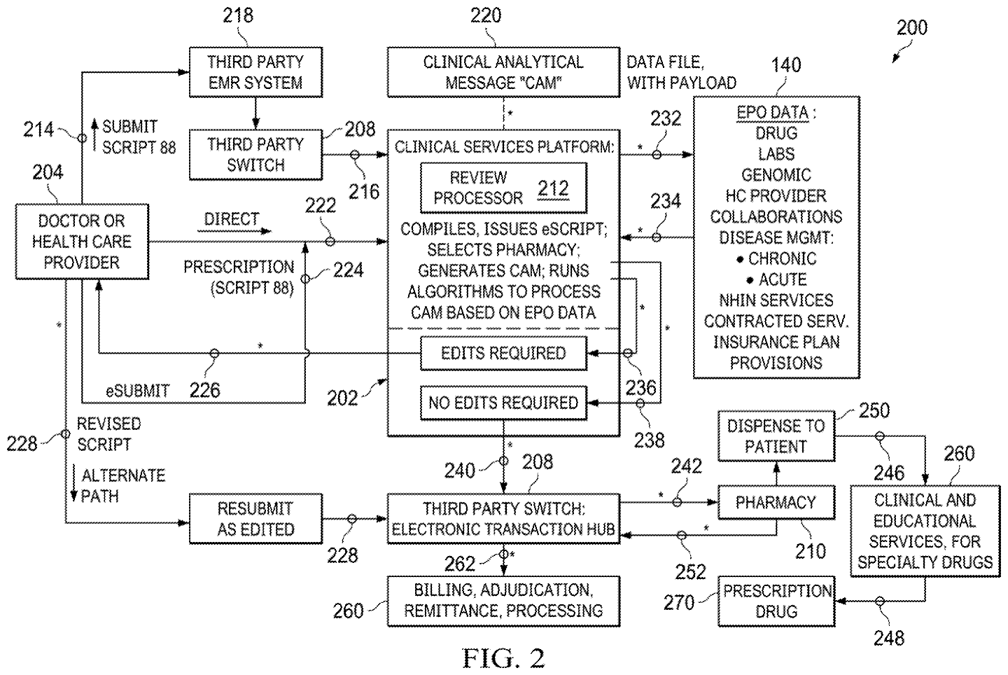

The use of a clinical analytical message (CAM) data file also provides an essential adjustment and/or confirmation function during the process of fulfilling a prescription. The term clinical means that the message includes the relevant known patient clinical data to help ensure that the supplied patient data is consistent with other information involved in the transaction taking place during the fulfillment process. The term analytical means that the drug data in the patient's file—description, genomic, interactions, and even laboratory data—that is specific to the patient for whom the prescription is being processed is analyzed for consistency with the clinical data that relates to the prescription. The term message means that the CAM includes relevant educational and clinical services information that must be delivered to the patient and to the electronic patient outcome record during the fulfillment process is readily available and communicated so that compliance with FDA and CMS requirements may be met during the steps of the fulfillment process. Details of the CAM data file are included in the description of FIG. 2 below.

One additional feature of the healthcare data system disclosed herein is the role of a patient specific algorithm (“PSA”) created for and unique to each patient. This algorithm functions as an address—a secure doorway—of the patient's file as it is accessed, retrieved, and updated by authorized users—the patient and authorized healthcare providers such as physicians, hospitals, pharmacists, laboratories, and payers. The PSA, which may be associated with a message header incorporated in the CAM data file, includes data security features that help prevent unauthorized access to the patient's data file.

FIG. 1 illustrates one aspect of the invention—a system that is structured to function like a healthcare utility. The system is configured to process data from a variety of sources in multiple categories. In one of the sources the EPO data includes but not is limited to patient demographics, individual patient medical information, genomics (including individual genetic data), disease profiles, allergy profiles, medications and immunizations, all of which are resident in the EPO data. In another source, the ePR database includes the pharmaceutical patient records of participating pharmacies. The data stored, accessed and processed by the system may also include laboratory data and analyses, and information regarding providers, payers, and appointment schedules and treatment. The operations of the system depicted in FIG. 1 will be described in further detail in the descriptions of FIGS. 2 and 3.

FIG. 1 illustrates a broad overview of the principal features of the system network for processing prescriptions for pharmaceuticals, particularly so-called “specialty drugs.” FIG. 2 illustrates the major process steps and functional components involved in a first embodiment—the front end processing of specialty drugs—the submission review phase associated with reviewing a prescription submitted by a physician or other practitioner for efficacy in light of comprehensive data stored in several databases—prior to submitting the prescription to a pharmacy for fulfillment. FIG. 3 illustrates the major process steps and functional components involved in a second embodiment—the back end processing of specialty drugs—the fulfillment review phase associated with delivering required clinical and educational services information to the patient and dispensing the specialty drug to the patient—following receipt of the prescription by the pharmacy. These reviews are necessary to ensure compliance with regulations and patient safety.

As will become apparent, an overall objective of the system of FIG. 1—is to utilize two of its key components—a comprehensive and innovative clinical services platform and an electronic patient outcome (EPO data) database—to create in combination with an electronic pharmacy record (ePR) database a novel, consolidated electronic patient outcomes record (EPOR) so that this unique EPOR patient record receives data updates in real time when processing submission and fulfillment of a submitted prescription that is sufficient to report, also in real time, measureable outcomes provided during an episode of care. It should also be apparent that this new EPOR is distinct from the two principal sources from which it is populated—the ePR and EPO databases. As the system described herein is installed and configured, connected through the Internet (or other network) to external entities and sources of data, the data necessary to populate the EPOR database may be translated to a common format compatible with the clinical analytical message (“CAM”) data file and stored in the EPOR database. Then, whenever the website portal is accessed and the system operated, updates to the EPOR and source data may take place through the operations of the clinical services platform to be described. In general, the structure and functional operations of the system that accomplish this objective are illustrated and described in FIGS. 2-5B.

On the left side of FIG. 2 is the healthcare provider who examines, diagnoses, and treats patients, often issuing prescriptions, and providing clinical information related to the prescribed treatment. Included on the right side is an extensive EPO data database, which may contain standardized files and services including but not limited to drug, laboratory, and genomic information plus data of healthcare providers, collaborations, disease management information concerning both chronic and acute conditions, and information regarding healthcare services. The software system, embodied in a clinical services platform identified with the pharmaceutical delivery system, is also coupled with the EPO data through an NHIN (National Health Information Network) system for supplying data and services to the EPO data. The pharmaceutical delivery system may also provide access via the protocols of X12 and NCPDP for processing billing for the delivery of clinical and educational services and for dispensing the specialty drug. The EPO data may also receive the data for e-scripts (electronic prescriptions) involved in editing of the prescription submission and fulfillment transactions (to properly align with payment requirements) that generally take place through an electronic transaction hub frequently identified as “the Switch.” The switch, a service that may be provided by any of several private entities, is a communications system configured for data interchange in the healthcare Network system to be described.

The central portion of FIG. 2 depicts, from top to bottom, two of the key components of the present embodiment—the CAM data file 220 and the clinical services platform 202, which together form a novel combination with the EPO data 140 not heretofore available. These key components together solve the problems of integration of the disparate components in the healthcare industry as described herein. A patient safety portal, which provides for safe and secure patient access to that patient's electronic patient outcomes record (EPOR) and provides the enabling authorization for clinical practitioners, is provided by the website portal 104 into the system 100 that facilitates much of the functionality of the present embodiment. The website portal 104 provides the interface into the interdisciplinary electronic patient outcomes record or EPOR from both the patient safety portal and interdisciplinary portals for each healthcare provider discipline shown coupled via the links 160 to the website portal 104 depicted in FIG. 1. Finally, also shown as coupled to the EPOR is the essential facilitating software system, called the specialized “clinical services platform” 202 for the operation of the EPO data 140 database. The software in the clinical services platform, including the review processor 212, is configured to interface with and interact with the pharmaceutical delivery system as will be described.

Two important constituents of the EPO data 140 are called a pre-clinical analytical message (“pre-CAM”) data file and a post-clinical analytical message (“post-CAM”) data file. These two data files contain standardized clinical requirements for each specialty prescription drug available in the system. This information, which may be verified by medical research institutions such as medical schools, independent medical research laboratories and the like, includes data about individual drugs and clinical education and clinical services information that must be delivered to the patient. The pre-CAM data file includes data about each drug and the required clinical education or clinical service information that must be delivered to the patient. The post-CAM data file includes notices that (a) informs the dispensing pharmacy when a drug can be ordered and dispensed; and (b) informs via the clinical services platform all providers involved in the patient's episode of care that the clinical education or other clinical services were performed.

It should be understood and appreciated that throughout the entire processes described herein, the data in each relevant database component—respectively the EPO data (the standardized files and services database), ePR data, and the EPOR database—associated with each process step is continually updated as an interaction with a provider or transaction or encounter with the system occurs. In particular, the system is structured to maintain the EPOR updated at all times so that the functions of subsequent steps may be available in real time through the portal links with the system that are provided in the system. Thus, the system enables—only as authorized—safe, secure access by the providers: physicians, hospitals, laboratories, payers, the CMS (Centers for Medicare & Medicaid Services, an agency of the Federal Government), and the patients themselves. The system may also provide for accessing one or more designated electronic patient outcome management portals and a business intelligence dashboard (BID) attached to the patient's EPOR in the system.

The system described herein is configured to process and manage the numerous healthcare data transactions associated with and that necessarily take place while diagnosing, advising, and treating a patient for an injury, disease, or disability. Such transactions are data intensive. Moreover, in such transactions, pharmaceutical prescriptions are, next to consultations, one of the most prevalent ingredients of the healthcare received by the patient. Thus, a data system organized around the process and management of the use of pharmaceuticals embodies a sufficiently broad perspective to use in devising a healthcare data system.

FIG. 1 illustrates a broad overview of the principal features of the system network for processing prescriptions for pharmaceuticals. FIG. 2 illustrates the major process steps and functional components involved in a first embodiment—the submission review phase associated with reviewing a prescription prior to submitting the prescription to a pharmacy for fulfillment. FIG. 3 illustrates the major process steps and functional components involved in a second embodiment—the fulfillment review phase associated with delivering required clinical and educational services information to the patient and dispensing the specialty drug to the patient.

A key feature of these embodiments is that a new consolidated Electronic Patient Outcome Record (EPOR) 150 consolidates data copied from two other databases (the EPO Data 140 and the ePR data 144) during processing of a prescription. The EPOR is maintained for access by any patient and practitioner, hospital and clinic, laboratory and medical research entity authorized to participate in the use of the system. The system is configured to update all database entries as each transaction takes place so that the data entry and access may be completed in real time.

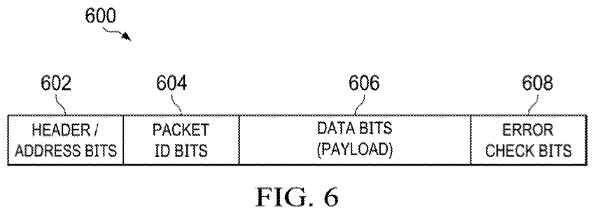

Another key feature of these embodiments is the use of a unique data file called a “clinical analytical message” or “CAM” data file that travels with the processing steps governed by software according to custom algorithms resident primarily in the clinical services platform. The CAM data file, which may be structured as depicted in FIG. 6 described herein, is used in both the submission review and the fulfillment review phases of the processing. These review phases are somewhat similar in concept but distinctly different in their structure and processes from the well-known “pre-edit” and post-edit” processes used by pharmacies and payers in the industry to examine prescription claims for accuracy and consistency using established data and rules for processing claims.

As mentioned previously, in the processing of prescriptions for specialty drugs it is required to supply clinical and educational services information to the patient who is to receive specialty drugs. As will be described, the information needed to satisfy that requirement will be stored in the EPO database 206, and the instruction regarding that requirement will be contained in the CAM data file, or CAM 220 in FIG. 2. In this case, the CAM 220 may be configured as a “Pre-CAM 220A” for “pre-clinical analytic message,” that governs the submission review phase of the prescription processing. The source of the clinical and educational services information is typically based on data verified by physicians and medical schools or medical research institutions.

Also associated with processing specialty drug prescriptions is a “Post-CAM 220B,” a post-clinical analytic message, that governs the fulfillment review phase of the prescription processing. The post-CAM 220B preferably includes, among other data, information about when the specialty drug can be dispensed by the pharmacy to the patient, or in some cases administered to the patient by a licensed healthcare practitioner, along with the required clinical and educational services information.

In the description that follows, a prescription, which is a unit of information, is given the reference number 88, and a claim, also a unit of information, is given the reference number 90. Two specific types of claims will be described: the claims 90 include first claims 91 for delivery of the clinical and educational services via path 312 and second claims 92 for dispensing the drugs.

FIG. 1 illustrates a system network diagram depicting major functional components of one embodiment of the present invention. The healthcare data system 100 is structured around its operational connections with the Internet 102, which is accessed via a website portal 104 and provides links with participating entities in the healthcare data system 100 via an electronic transaction hub 106. The electronic transaction hub 106 is known in the industry as the “switch” and will be referred to herein as the switch 106. Coupled to the Internet via first 112 and second 122 data processing links are first 110, second 120, and third 130 “cloud” systems. The cloud systems 110, 120, and 130 are shown as three separate entities because they each may contain the same data and processing and programming structures to provide essential redundancy and back up capability.

As is well known, a cloud may be defined as a system of computing resources—data storage, data processing, software, and communications components remote from user locations but accessible to the users on request. One principal utility of data processing facilities available in clouds is that users are freed of the burdens of installing and maintaining their own data processing resources. Instead, the necessary data processing components, including proprietary software stored in a cloud, can be accessed at will during the operation of the user's systems. Thus clouds may reduce the capital expense of maintaining one's own computing infrastructure to an operating expense. Another important advantage of clouds is the ease of providing redundant and secure resources for critical systems such as healthcare data systems that have substantial data backup and security requirements. Clouds may be public or private. Public clouds may have multiple users who share the available resources. Private clouds are maintained by individual entities that limit access for their own purposes.

In FIG. 1, the cloud 110 includes a first review processor 114, which may be proprietary software that fulfills an important function in the healthcare data system 100. Similarly, the cloud 120, a duplicate of cloud 110, provides system redundancy and contains duplicate proprietary software, a second review processor 124. To illustrate that certain important segments of healthcare data are stored in the first 110 and second 120 clouds, a third cloud 130 is depicted in FIG. 1, which represents respective portions of the first 110 and second 120 clouds. The data storage functions of the third cloud 130 are shown accessible through a third data processing link 132 that connects them with the website portal 104. Also connected to the third data processing link 132 are the three databases: EPO Data 140, ePR Data 144, and the consolidated EPOR 150.

The EPO data 140 may be a database containing standardized files and data and services that may include but not be limited to 1) drug data; 2) laboratory data; 3) genomic data; 4) healthcare provider data; 5) data on collaborations among healthcare providers; 6) disease management information for both chronic and acute conditions; and data about 7) NHIN services and contracted services. The EPO data may be accessed by public participants in the healthcare data system 100.

The ePR data 144, named an “electronic pharmacy record, may be a central, intra-pharmacy database that stores patient and pharmaceutical data associated with prescription processing by participating pharmacists.

The EPOR 150 may be a consolidated database structured and dedicated for use by participating members of the healthcare data system 100. It receives data and information updates with each healthcare transaction that takes place in and processed by the system 100. Data and information are received from both the EPO data 140 and from the ePR data 144, which are not generally accessible to all providers. Thus, whenever one of the participating providers authorized to log into the website portal 104 via the respective portals 162-174, the data accessible to them resides in the consolidated database called EPOR data 150.

Two groups of participants in the healthcare data system 100 are represented in FIG. 1. One group is coupled via provider portal links 160 to the website portal 104. This group of participants includes, but is not necessarily limited to patients 162, physicians 164, pharmacies 166, hospitals and clinics 168, laboratories 170, and entities providing genomic research 174. Providers of genomic data 172 and medical school data 176 may be linked respectively to the laboratories 170 and the research entities 174. Another group of participants includes payers and industry standard mechanisms 180, which is coupled to the electronic transaction hub 106 (aka the switch 106) through a switch link A 182. The payers and industry standard mechanisms 180 is also coupled to a National Health Information Network or NHIN 142 via a switch B link 184. The NHIN 142 receives data from the payer and industry standard mechanisms 180 via the switch link B 184. The payer and industry standard mechanisms 180 provides for processing claims for payment of services rendered by healthcare providers including the participants of the first group connected via provider portal links 160 to the website portal 104.

One other principal participant in the system 100 may be a network of pharmacies 146, which accumulates massive amounts of patient, pharmaceutical and other medical information involved in processing prescriptions and claims for payment. This healthcare data is available to supply both the NHIN 142 and the ePR 144 with up-to-date information about each electronic healthcare transaction processed by members of the network of pharmacies 146 via the respective fourth 138 and fifth 148 data links to the NHIN 142 and the ePR 144.

Either of the first and second review processors 114 or 124, which may reside in the first and second clouds 110, 120, fulfills a central role during submission processing of prescriptions submitted into the healthcare data system 100 by a healthcare provider. The review processors 114, 124, collectively represented in FIG. 2 by review processor 212, may preferably be a functional part of the clinical services platform or a distinct part of the clinical services platform that is directly integrated with the clinical services platform. As described herein for convenience, it will be considered as part of the clinical services platform.

FIG. 2 illustrates a functional block diagram of front end processing of a prescription for a so-called “specialty drug” according to one embodiment of the invention. The term “front end” processing refers to submission processing of the prescription for the specialty drug. So-called “specialty drugs,” are typically very expensive, require special handling, and prescribed for rare or debilitating diseases. The submission processing affects handling of the prescription—i.e., evaluation of the prescription in an automatic editing process—from entry of the prescription into the system by a healthcare provider (such as a physician) of a prescription for a patient to reception by a pharmacy selected to fulfill the prescription by dispensing the drug to the patient or a caregiver.

FIG. 2 depicts an example of the “front end” processing of a pharmaceutical prescription. The processing by submission review system 200 performs review of a prescription 88 submitted by a healthcare provider 204 such as a physician or nurse practitioner in real time via either of the paths 214, 222, or 224. Thus, when the prescription 88 is submitted by hitting the send key on the provider's terminal, the system 200 performs the submission review and returns, within a few seconds, either a confirmation that no edits to the prescription 88 are required or an instruction that the prescription 88 must be revised because of one or more factors that turned up during the submission review process that would invalidate the initial prescription 88 for its intended purpose. In the following description, a prescription 88 is defined to be a unit of information identifying a drug prescribed by a provider, when to administer the drug (including how often during each 24 hour period), and the quantity of the medication (typically specified in milligrams or “mg.”). This “script” information is used by the dispensing pharmacy to fill the prescription 88 and by the patient as the instructions in administering the drug.

The submission processing system 200 shown in FIG. 2 includes a review processor 212 in a clinical services platform 202, a licensed healthcare provider 204, an EPO database (“EPO data”) 140, an electronic transaction hub (or “switch”) 208, and a pharmacy 210, all coupled through communication links as will be described. The review processor 212 may be a system of software applications operating on a computer or server system, on user infrastructure or preferably, remotely disposed in a cloud 110, 120, 130 as depicted in FIG. 1. A licensed healthcare provider 204 may be a physician or medical doctor, a nurse practitioner, either in private practice or in a hospital or clinic and the like. An EPO data 140 database may be a part of the user's infrastructure or, preferably, a portion of a remote resource such as the cloud 110, 120, 130. An electronic transaction hub or switch 208, typically provided by a third party, appears in the communication path 214 between the licensed healthcare provider 204 and the review processor 212 in the clinical services platform 202, as well as the communication path 240 between the review processor 212 and the EPO data 140. In the following description the term “path” is understood to mean “communication path.”

It will be noted that there are several communication paths available as alternates in cases where editing of the prescription is required. For example, a prescription 88 may be submitted through a third party electronic medical record system (“EMR”) 214 and the third party switch 208 to an input of the review processor 212 in the clinical services platform 202. Alternatively, the prescription 88 may be submitted directly via direct route 222 or an eSubmit route 224. Upon receipt, the review processor 212 compiles the information in the prescription 88, and may select a pharmacy based on information in its databases about the patient and the type of medication. The review processor 212 generates a unique type of data file called a clinical analytical message (“CAM”) data file 220 that travels through the system with the prescription 88. As illustrated in FIG. 6, the CAM data file 220 may include a data payload having patient data fields, a routing field or header, an error check field, and a packet identifier used as a key during the pre-edit processing. The payload in the CAM data file 220 may function like a checklist associated with the prescription as it works its way through the submission review processor. The CAM data file 220 is exchanged during submission processing between the functional units shown in FIG. 2 along the data links marked with an asterisk (*). The use of double asterisks (**) in FIG. 3 denotes the use of the CAM data file 220 during fulfillment processing.

The clinical services platform 202, which includes the review processor 212, comprises a review (or, alternatively, a pre-edit) processor operative on the CAM data file 220 contents and with the databases. The review processor may preferably include a suite of pre-editing algorithms for accessing the patient clinical data in the CAM data file, analyzing the patient clinical data in the context of the submitted prescription and information about the prescribed pharmaceuticals, and completing the pre-editing process.

The review processor 212 may further comprise a first process for automatically performing pre-edit review of the submitted prescription and calling in sequence individual ones of the pre-editing algorithms to access, retrieve, and analyze the patient clinical data according to available information about the drug identified in the submitted prescription; a second process for controlling the plurality of communication links called during the pre-editing process; a third process for selecting a pharmacy for fulfillment of an edited prescription based on pharmacy authorization data stored in the database corresponding to a prescribed drug identified in the edited prescription; and a fourth process for automatically performing the steps required to fulfill the submitted prescription including delivering required clinical and educational services and dispensing the prescribed medication. The review processor 212 may preferably include an interface operable for sending the electronic prescription as a single synchronous message.

In the illustrated embodiment, the clinical analytical message data file 220 (CAM data file) contains (A) information resident in the patient data fields for completing drug utilization review (“DUR”), including at least (1) a plurality of patient clinical data retrieved from addressable locations in the database associated with the patient and the submitted prescription; (2) statements reporting results of analysis of the submitted prescription against patient clinical data retrieved from the database and analyzed by the software mechanism; and (3) information regarding required clinical education and services to be delivered to the patient with the dispensed prescription. The CAM data file may also contain (4) a structure for cooperating with one or more pre-edit algorithms to perform analytical processing of stored patient clinical data to determine consistency [efficacy of] with the submitted electronic prescription, wherein the structure for cooperating may include a processing key associated with the one or more of the pre-edit algorithms.

The processing speed that enables the submission review system 200 to respond to the provider in real time is provided by the combination of dedicated software located within a virtual host or cloud and operated when called within the review processor 212 during submission review and upon direct access from the review processor 212 to the comprehensive EPO data 140 database. Having the wide range of patient, pharmaceutical, and insurance plan information assembled in one electronic location—the EPO data 140—and directly accessible by the review processor enables the dedicated software to perform the submission review processing with practically zero delay.

The sequence of operations typically begins when a healthcare provider 204, through a computer terminal (a ubiquitous device not shown), enters an eScript or prescription 88 for a drug to be administered to a patient. The entered prescription 88 may be transmitted directly to the review processor 212 of the illustrated submission review system 200. Alternatively, the prescription 88 may be submitted through an existing third party EMR system 218 via the switch 208 to the review processor 202, or the prescription 88 may be submitted electronically as shown. The switch 208 (which may be called a gate) may be any of several telecommunications network and data formatting services that facilitate transmission of prescription information among participating entities connected in the network 102.

Operations performed in and by the submission review processor 202 include recording the prescription information entered by the provider 204 with the identity of the patient's preferred pharmacy, and assembling the CAM data file 220 by populating its data fields with relevant data from the EPO data 140, an action that is analogous to filling out a form, albeit nearly instantaneously. Using the file tag, the review processor 212 calls custom algorithms to analyze the populated data fields in the CAM data file 220 in light of the prescription 88 entered into the submission review system 200. The algorithms are constructed to perform drug utilization reviews, analyzing the prescription for drug interaction with information in the EPO data 140 database such as (but not limited to) other drugs the patient may be taking, known food or other allergies, particular disease conditions of the patient, laboratory data from various tests (e.g., blood, urine, and other body fluids), patient disease and medical data, the patient's genomic profile, and provisions of the patient's health insurance that may be relevant to the particular prescription.

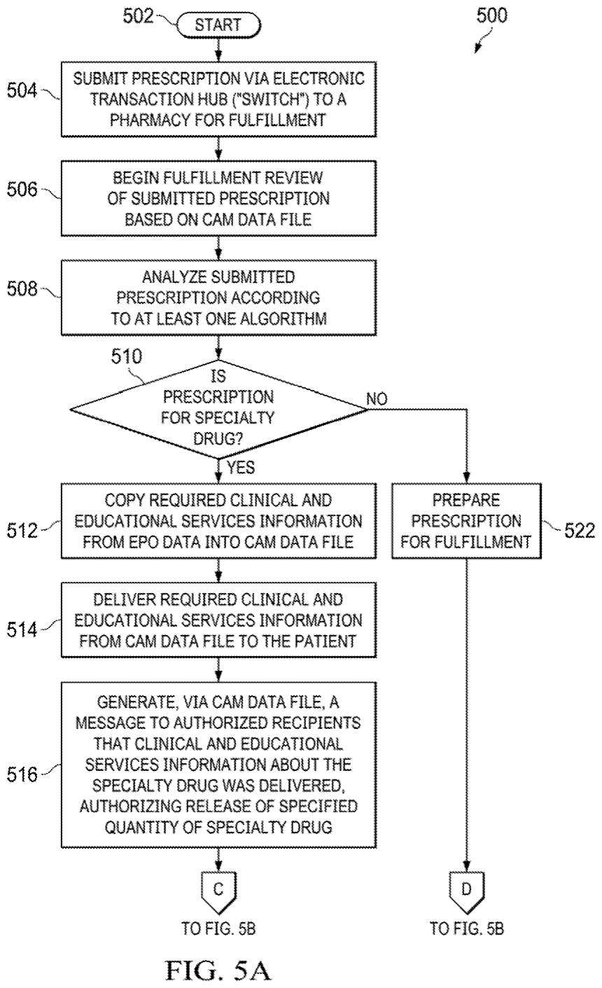

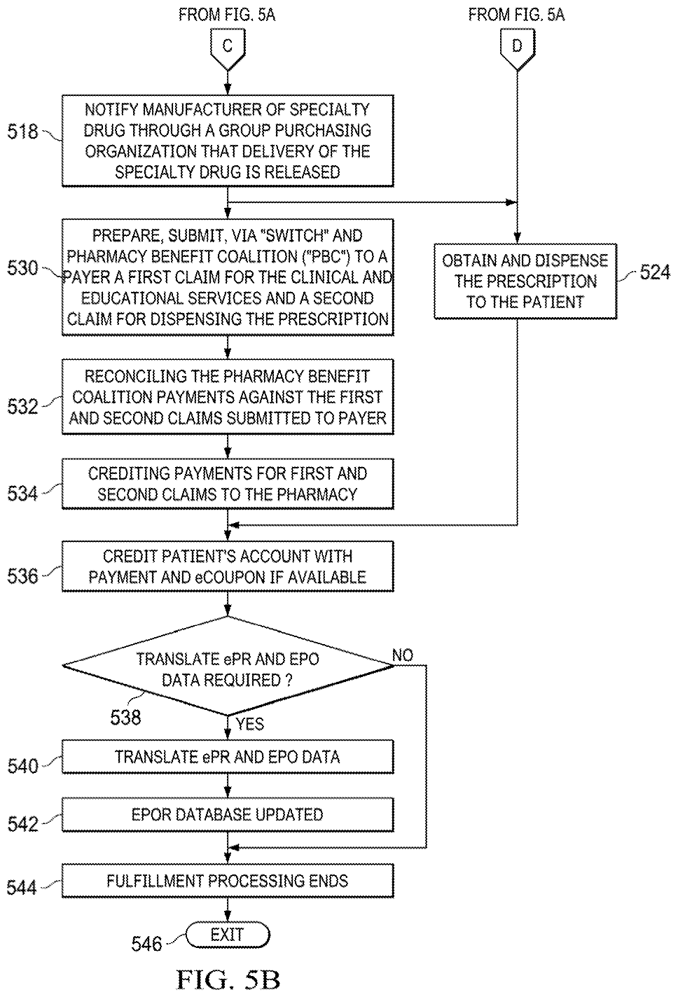

The operations performed by the review processor 212 determine whether the submitted prescription 88 must be edited to correct an incompatibility or inconsistency of the prescribed drug with some item of information in the EPO database 140. The result is either an instruction 224 returned to the provider 204 to revise the initial prescription 88, along with a suggestion for the needed revision; or the prescription 88 is forwarded 228 to the pharmacy 210 via the switch 208. In some systems a message (not shown in FIG. 5A) that no editing is necessary may be returned to the provider 204 to confirm forwarding of the prescription to the pharmacy 210. In the case of a prescription needing revision, the provider may be given the opportunity to make the suggested revision and resubmit a revised prescription to the pharmacy 210 via the switch 208. Upon receipt, the prescription 88 may be placed in a queue for fulfillment by the pharmacy 210.

In many cases fulfillment—sometimes called “back end processing”—simply means dispensing the prescription to the patient, and accounting for payment by the patient's insurance carrier. In other cases, such as for specialty drugs—medicines that require administration by a licensed practitioner, or chemotherapy drugs, or experimental drugs, or highly expensive drugs—the fulfillment process is far more complicated as will be described herein with FIG. 3. For example, a Federal Government requirement imposed on the fulfillment process by CMS, the Center for Medicaid and Medicare Services, is the need to record data regarding the outcome of the prescription process—i.e., whether the data stored in the EPO data 140 (the standardized healthcare data) and the supplementary database called the EPOR database 150 (FIG. 1) or 304 (FIG. 3) (that is accessible to patients and healthcare providers) reflects a proper outcome of the process, that all requirements have been satisfied. The consolidated EPOR database is populated with data extracted and translated, by means well known in the art, from the typically disparate formats used in the ePR and EPO databases, for storage in a single database with all data formatted in a common format for ready access by authorized providers and the patients.

FIG. 3 illustrates a functional block diagram of back end processing of the prescription 88 for a specialty drug according to the embodiment of FIG. 1, and follows and is closely related to the embodiment of FIG. 2. Portions of FIG. 2 appear in FIG. 3; accordingly those structures of FIG. 2 bear the same reference numbers. For example, the switch 208 is shown with an input from the review processor 202, which in turn is coupled and has access to the contents of the databases shown in FIG. 2, namely: the EPO data 140, the ePR data 144, and the EPOR data 150. The term “back end processing” refers to the fulfillment processing of the prescription 88. The system is specifically configured for processing prescriptions 88 for specialty drugs. The fulfillment processing affects handling of the prescription 88 by the pharmacy beginning with receipt of an edited version of the prescription 88 submitted from the originating healthcare provider. The process of fulfillment includes providing clinical and educational services information to the patient, acknowledging that service, filling and dispensing the prescription drug to the patient, preparation of claims 90 for payment for those services, and processing of those payments to the manufacturer, distributor, and retail pharmacy by the payers on behalf of the patient.

The fulfillment processing begins when a prescription 88 (originated by a healthcare provider 204) sent from the review processor 202 with the CAM data file 220 is received via path 212 and transmitted by the switch 208 over path 242 to the pharmacy 210. The pharmacy 210 performs several functions. If the prescription 88 is for a specialty drug the pharmacy 210 reviews and prepares the prescription 88 for delivery of clinical and educational services at step 260 along paths 330 and 334 and dispensing or administering the prescription drug to the patient in step 302 through the step 250. The clinical and educational services information is contained in the CAM data file 220 that travels with a specialty drug prescription. Upon delivery of these services via step 260, the CAM data file 220 confirms delivery of the services 260 along path 262. From there the message travels via step 340 along path 264 via the switch 208 to update the EPO data 140 thereby notifying the healthcare provider 204, and also to the manufacturer 360 of the specialty drug via the path 254, thereby notifying the manufacturer that the order for the specialty drug sent by the pharmacy 208 over the path 332 can be released. If the prescription is not for a specialty drug, no clinical or educational services are required and the pharmacy 210 dispenses the drug to the patient 302 at step 250.

Fulfillment processing includes processing of claims 90 for payments to the pharmacy 210 for services it renders. It also includes processing of payments to the manufacturer 360 of the specialty drugs after it delivers the drugs to the inventory of a Group Purchasing Organization or structure 370 (aka GPO 370″). The GPO 370 is established to serve a warehouse function by member pharmacies to maintain an inventory of specialty drugs and facilitate processing of payments and credits for its costs. The specialty drugs received from the manufacturer 360 along path 342 are delivered by the GPO 370 after the specialty drug manufacturer 360 receives the order from the pharmacy 210 along path 332. Confirmation of delivery 340 of the clinical and educational services to the patient via path 264 authorizes release of the specialty drug by the manufacture 360 via path 342 to the GPO 370, which delivers the specialty drug to the pharmacy along path 348.

Similarly, the path 342 represents the dual role of delivering, to order, the specialty drug from the manufacturer 360 to the GPO 370, and for sending the bill for the specialty drug to the GPO 370. Associated with and in response to receipt of the bill for the specialty drug by the GPO 370, followed by remittance of payment for the specialty drug along path 344 to the manufacturer 360, the manufacturer 360 may issue an electronic coupon (aka “eCoupon”) via path 346 that represents a discount to be credited to the patient's account at the pharmacy 210 for the cost of the specialty drug. The eCoupon 346 is processed by a Pharmacy Benefit Coalition or entity (aka “PBC”) 310 en route to the GPO 370 for delivery to the pharmacy 210. The PBC 310 acts as a coordinating entity for pharmaceutical benefits available from payers associated with the patient—usually an insurance company or employer by contract or benefit plan or Federal or State benefits such as Medicare and Medicaid.

The pharmacy benefit coalition or PBC 310 is an entity formed by the inventor of the present system as an efficient alternative to the industry's pharmacy benefit manager (or “PBM”). The PBC is devised to efficiently coordinate, adjudicate as necessary, and reconcile the payer benefits to the pharmacy on behalf of the patient by clearing e-prescriptions (“eRx”) for fulfillment and facilitating the transactions associated with fulfilling the e-prescription. The GPO 370 may also forward payment for the specialty drug to the manufacturer 360 via the path 344.

When the pharmacy 210 has delivered the clinical and educational services (for specialty drugs) and dispensed the specialty of non-specialty prescription drugs, and established acknowledgement of those services, the pharmacy 210 prepares and files claims 90 for payment with the PBC 310. The claims 90 include first claims 91 for delivery of the clinical and educational services via path 312 and second claims 92 for dispensing the drugs. The PBC then forwards the claims 91, 92 to a payer of record 320, which may adjudicate the claims 91, 92 according to its own rules and procedures before forwarding them to the PBC 310 to reconcile the required remittances and advise the PBC 310 as necessary before submitting the reimbursements along the path 324 to the pharmacy 210. The processing of these claims 91, 92 may also be characterized respectively as the first and second reimbursement transactions.

Persons of skill in the art will recognize that the system described herein forms a new combination of new structures with existing structures operative in the healthcare industry. The new system helps providers comply with Federal mandates to provide a safe, efficient, and secure patient outcomes record that enables objective measurement of the outcome of episodes of care received by patients. The new structures include a system of an associated electronic patient outcomes record (EPOR), a new clinical services platform, and a new clinical analytical message data file. The system is organized around the systems, processes, and databases for prescribing and verifying (submission), and fulfilling drug prescriptions, providing clinical data and counseling, reconciling the associated payment transactions, and providing real time updates to the stored data at every step of the processes. Structural features of the system are linked through individual portals with healthcare providers and which also permit access by individual patients through a dedicated, secure portal into the system and the electronic patient outcomes record (EPOR). These structural features include a variety of new software mechanisms within the clinical services platform, a National Health Information Network (NHIN), and a pharmaceutical delivery system that are configured for communication such that portability and interoperability of the all healthcare-related data in the system is ensured.

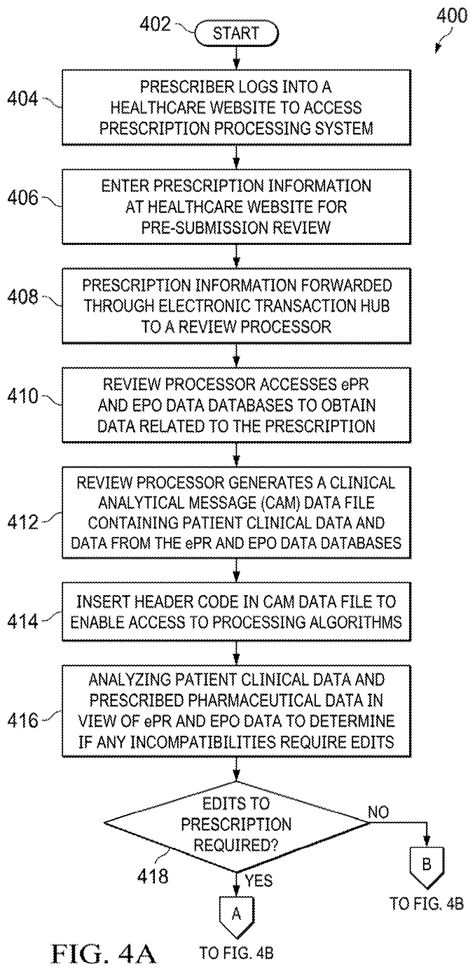

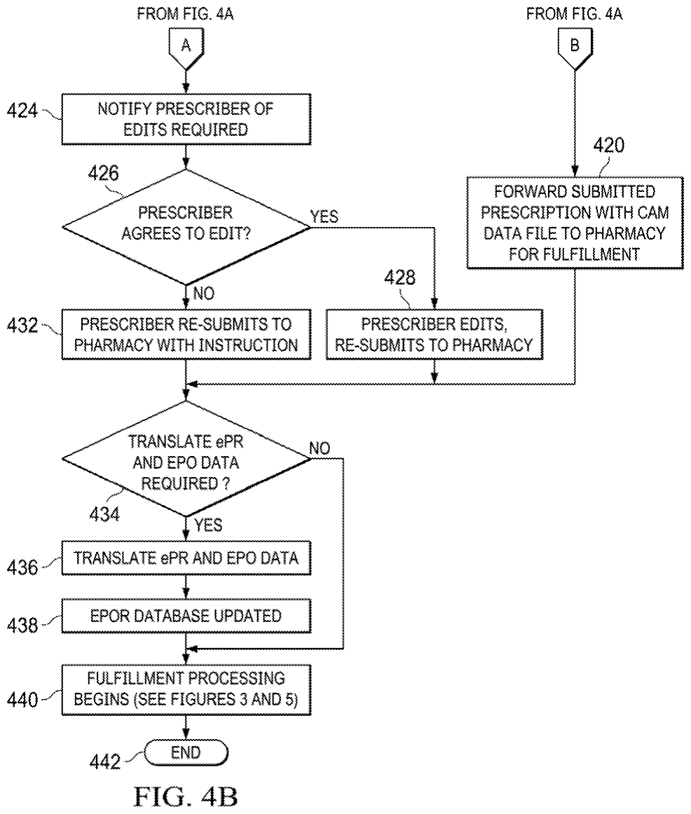

To provide further description of the submission and fulfillment processes, flow charts of these functions, illustrated in FIGS. 4A, 4B, 5A, and 5B will now be described. FIG. 4A illustrates a flow chart diagram of a first portion of the front end or “submission” processing 400 performed by the system depicted in FIG. 2. Starting at block 402, the flow begins when a prescriber such as a licensed healthcare provider 204 logs into a healthcare website 104 to access a prescription processing system 100 in step 404. Upon a successful log-in the provider 204 enters prescription information at the healthcare website 104 to initiate a pre-submission review in step 406. In the following step 408 the prescription information is forwarded through an electronic transaction hub or “switch” 106 to a review processor 202. The review processor in step 410 accesses data in the EPO database 206 to obtain data related to the submitted prescription 88. In step 412 the review processor generates a clinical analytical message or “CAM” data file 220 containing patient clinical data and data obtained from the EPO database 206. Generation of the CAM data file is followed by step 414 to insert a header code in the CAM data file 220 to track the prescription during the submission process and enable it to be accessed by processing algorithms in the review processor 202.

Advancing to step 416, the review processor analyzes the patient clinical data and the prescribed medication in the prescription 88 in view of the EPO data 206 to determine if any incompatibilities exist that require edits to the prescription 88. Step 418 responds to the determination by directing the flow depending on whether edits to the prescription 88 are or are not required. If edits are not required, the flow proceeds via (B) to step 420 in FIG. 4B. If edits are required, the flow proceeds via (A) to step 424 in FIG. 4B.

FIG. 4B illustrates a flow chart diagram of a second portion of the front end or submission processing 400 performed by the system 100 depicted in FIG. 2. From step 420, which forwards the submitted prescription 88 with the CAM data file to a pharmacy 210 for fulfillment. The fulfillment processing begins in step 430 as will be described using FIGS. 3 and 5. From step 424, which notifies the prescriber 204 what edits are required to the prescription 88, the flow advances to a decision step 426 in which the prescriber decides whether to agree to the required edits or not to agree to these edits. If the prescriber agrees to make the edits, the edits are made and the prescription is resubmitted in step 428 to the pharmacy 210 so that fulfillment can begin in step 440. If the prescriber disagrees with the requirement to edits the prescription, the prescriber resubmits the prescription 88 in step 432 with an instruction and or explanation that the prescription as originally issued is required by the diagnosis and an exception must be made to the required edits. If the analysis determined that no edits were required in step 418 (FIG. 4A) the flow proceeds to step 434. Following steps 420, 428 and 432 the flow advances to a step 434 to determine if translation of the ePR and EPO data from its native format is required. If no, the flow advances to step 440 to begin fulfillment processing as described in FIGS. 3 and 5. If step 434 is yes, the source data (ePR, EPO) must be translated, the flow is directed to step 436 to translate the source data from its native format to the common format utilized by the system database, the EPOR. Thereafter the EPOR is updated and the process enters the fulfillment process at the step 440 and the submission process 400 ends at step 442. Thereupon the flow advances to step 440 to begin the fulfillment processing of the unedited prescription.

FIG. 5A illustrates a flow chart diagram of a first portion of the back end or “fulfillment” processing 500 performed by the system depicted in FIG. 3. Starting at block 502, the flow begins when a prescription 88 is submitted through an electronic transaction hub or “switch” 208 to a pharmacy 210 for fulfillment in step 504. In step 506 the fulfillment review process of the submitted prescription 88 begins based on the prescription information and the CAM data file 220. In step 508 the submitted prescription is analyzed according to at least one algorithm in the review processor 202. In the following step 510 the determination is made whether the submitted prescription if for a specialty drug and if the test is negative the flow proceeds in step 522 to step 524 in FIG. 5B. If the test in step 518 is positive, that is, if the prescription submitted for fulfillment is a specialty drug, then the flow proceeds to step 512 where the clinical and educational services information required for the specialty drug is retrieved or copied from the EPO data file and inserted into the CAM data file 220. In the following step 514 the clinical and educational services information is delivered to the patient. Then, in step 316 a message is generated using the CAM data file 220 to the authorized recipients that the clinical and educational and services information about the specialty drug was delivered to the patient, thereby authorizing the release of the specified quantity of the specialized drug by the manufacturer.

Turning now to FIG. 5B there is illustrated a flow chart diagram of a second portion of the back end or fulfillment processing 500 performed by the system depicted in FIG. 3. For a specialty drug the next step is to notify the manufacturer of the order for the specialty drug through notice to the Group Purchasing Organization or GPO 370 (FIG. 3) that delivery of the prescription drug is released since the clinical and educational services have been delivered (step 514) by the pharmacy to the patient 302. In step 524 the prescription drug, whether a specialty drug or is not a specialty drug is dispensed to the patient 302 by the pharmacy. In step 530 the pharmacy prepares and submits claims for payment to the payer 320 in the system by submitting via the electronic transaction hub or “switch” 208 and the pharmacy benefit coalition or “PBC” 310 to a payer 320 a first claim for the clinical and educational services it delivered and a second claim for the prescription drug it dispensed to the patient 302. In the next step 532 the payer may perform adjudication according to its rules and procedures to reconcile the payments by the PBC 310 with the first and second claims submitted to the pharmacy 210. When the payments and claims have been reconciled as needed the payments are forwarded to the pharmacy 210 in step 534 and credited, along with the eCoupon, to the patient's account in step 536. In the next step 538, the system determines whether the ePR and the EPO data databases need to be translated. If not, then the flow proceeds to step 544 and the fulfillment processing ends. If the ePR and EPO data must be translated from their native format, determined in step 538, the flow proceeds to step 540 to translate the ePR and EPO data into the common format for the EPOR database. After the translation step the EPOR database is updated and the flow advances to step 544 and the fulfillment processing ends, followed by exit from the fulfillment processing operations in step 546.

FIG. 6 illustrates one example of a data bit structure that may be used in one embodiment of the invention. The CAM data file structure used in the present invention may take any of several forms depending on the particular communications protocol employed in the system. The example illustrated in FIG. 6 defines a data file as a set of data packets, each packet being a sequence of data fields according to the kind of data encoded therein. Each data field 600 consists of frames of data. The example depicted in FIG. 6 includes, reading from left to right, a header or address field 602, a packet ID field 604, a data field (the data payload) 606, and an error check field 608. The fields of data are composed in frames of data made up of data bytes.

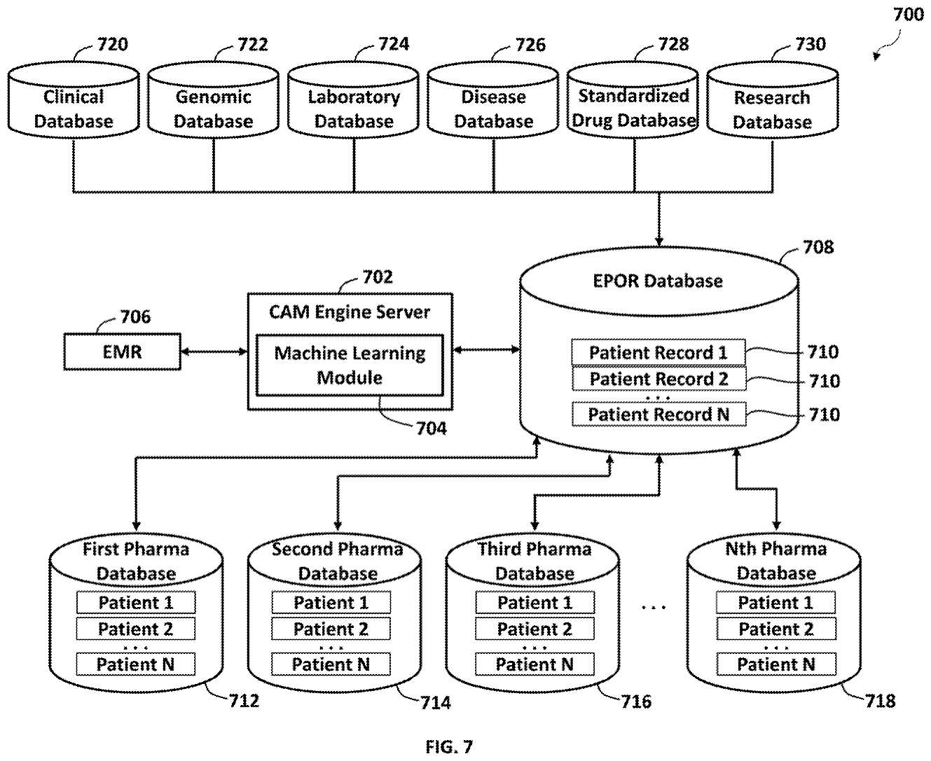

FIG. 7 illustrates a network diagram of functional system components depicting another embodiment of the present invention. A healthcare data system 700 can include a CAM engine server 702, an electronic medical record system (“EMR”) 706, an EPOR database 708, a plurality of patient databases 712, 714, 716, 718, a clinical database 720, a genomic database 722, a laboratory database 724, a disease database 726, a standardized drug database 728, and a research database 730.

The CAM engine server 702 is preferably implemented in hardware, software, or a suitable combination of hardware and software thereof and may comprise one or more software systems operating on one or more servers, having one or more processors, with access to memory. Server(s) can include electronic storage, one or more processors, and/or other components. Server(s) can include communication lines, or ports to enable the exchange of information with a network and/or other computing platforms. Server(s) can also include a plurality of hardware, software, and/or firmware components operating together to provide the functionality attributed herein to server(s). For example, server(s) can be implemented by a cloud of computing platforms operating together as server(s).

Memory can comprise electronic storage that can include non-transitory storage media that electronically stores information. The electronic storage media of electronic storage may include one or both of system storage that is provided integrally (i.e., substantially non-removable) with server(s) and/or removable storage that is removably connectable to server(s) via, for example, a port (e.g., a USB port, a firewire port, etc.) or a drive (e.g., a disk drive, etc.). Electronic storage may include one or more of optically readable storage media (e.g., optical disks, etc.), magnetically readable storage media (e.g., magnetic tape, magnetic hard drive, floppy drive, etc.), electrical charge-based storage media (e.g., EEPROM, RAM, etc.), solid-state storage media (e.g., flash drive, etc.), and/or other electronically readable storage media. Electronic storage may include one or more virtual storage resources (e.g., cloud storage, a virtual private network, and/or other virtual storage resources). Electronic storage may store software algorithms, information determined by processor(s), information received from server(s), information received from computing platform(s), and/or other information that enables server(s) to function as described herein.

Processor(s) may be configured to provide information processing capabilities in server(s). As such, processor(s) may include one or more of a digital processor, an analog processor, a digital circuit designed to process information, an analog circuit designed to process information, a state machine, and/or other mechanisms for electronically processing information, such as FPGAs or ASICs. The processor(s) may be a single entity, or include a plurality of processing units. These processing units may be physically located within the same device, or processor(s) may represent processing functionality of a plurality of devices operating in coordination or software functionality.

The processor(s) can be configured to execute machine-readable instruction or learning modules by software, hardware, firmware, some combination of software, hardware, and/or firmware, and/or other mechanisms for configuring processing capabilities on processor(s). As used herein, the term “machine-readable instruction component” may refer to any component or set of components that perform the functionality attributed to the machine-readable instruction component. This can include one or more physical processors during execution of processor readable instructions, the processor readable instructions, circuitry, hardware, storage media, or any other components.

The CAM engine server 702 can include a machine learning module 704. The machine learning module 704 can be implemented on one or more servers, having one or more processors, with access to memory. The machine learning module 704 can be a single networked node, or a machine learning cluster, which can include a distributed architecture of a plurality of networked nodes. The machine learning module 704 can include control logic for implementing various functionality, as described in more detail below. The machine learning module 704 may, based at least partly on a patient's information, conduct a patient analysis to establish a profile for the patient. The patient profile can correspond to health concerns based at least in part on the identified patient's pharmaceutical, clinical, genomic, laboratory, disease, or standardized drug information. Such information can include parameters or fields related to specific attributes of the information. A patient's health information or parameters can include patient demographics, individual patient medical information, ethnicity, genomics (including individual genetic data), disease profiles, allergies, allergy profiles, immunizations, blood type, weight, height, pre-existing medical conditions, metabolic rate, current medication usage, blood pressure, red blood cell count, white blood cell count, cholesterol levels, insurance information, drug side effects, and drug cost, among other information or parameters.

The machine learning module 704 can conduct an analysis of the patient's information (record) and/or parameters to generate a health concern level for a patient, such as low, medium, high, or a combination and store the level in a database. In some embodiments, the machine learning module 704 can update the health concern level for a patient periodically, based on updated information, as the patient's information or parameters change. The machine learning module 704 can also assign the patient a unique identifier process data associated with to conduct further analysis on the patient, based on information such as account information, account history, and loyalty program membership, among other relevant information. The machine learning module 704 can generate one or more health concern definitions, including one or more health concern parameters related to the patient information and standardized drug data. The health concern definitions can establish thresholds for specific parameters. The definition thresholds can be binary (present or not present), to indicate whether a condition exists, such as an allergy to a medication (e.g., penicillin, etc.), a food allergy (e.g., dairy, peanut, etc.), whether a prescribed drug would have a negative interaction with a drug the patient is currently taking, among others. The definition thresholds can also have minimum or maximum values for parameters having a scale, magnitude, or degree, such as a metabolic rate, a body mass index value, a weight, a height, a white blood cell count, among other parameters.

The machine learning module 704 can, based on the patient information or parameters, conduct a predictive analysis on a user. The predictive analysis can indicate whether a particular drug will have a negative interaction with a patient, whether a patient prefers generic drugs or name-brand drugs, whether a patient is likely to seek a refill, among other predictive analysis. The predictive analysis can be based in part on the patient's history or a patient's indicated preferences. The predictive analysis can further include personalized recommendations, such as bundles of related drugs or treatments.

The electronic medical record system (“EMR”) 706, can be a prescription entry system, an access portal to a patient's health information, or other suitable system. In one exemplary embodiment, a doctor can generate an electronic prescription or “eScript” for a drug to be administered to a patient. The eScript can include fields or parameters related to specific attributes of the prescription, including a drug identifier, a dosage amount, or a dosage frequency, among other drug attributes.

An electronic pharmacy record (“ePR”) data repository can include a plurality of patient databases 712, 714, 716, 718. Each of the plurality of patient databases 712, 714, 716, 718 can be configured to store pharmaceutical information associated with each of a plurality of patients associated with a first entity. The first entity can be a pharmacy chain, a particular pharmacy, a hospital, a doctor's office, or other suitable entity. The pharmaceutical information associated with each of a plurality of patients can contain entries, fields, or parameters associated with information related to the patient. The patient entries in the plurality of pharmaceutical databases can be accessed using identifying information for an identified patient.

The clinical database 720 can be configured to store clinical information associated with each of a plurality of patients. The clinical database 720 can be accessed via a local area network, wide area network, application programming interface, or other suitable communication mechanism. Clinical information can include information obtained during a visit to a doctor, hospital, clinic, or the like, for a specific patient, such as height, weight, blood pressure, symptom description, medical history, family history, or other relevant information.

The genomic database 722 can be configured to store genomic information associated with each of a plurality of patients. The genomic database 722 can be accessed via a local area network, wide area network, application programming interface, or other suitable communication mechanism. Genomic information can include information related to a particular patient's DNA, RNA, ChIP, MDB, or genes, generally.

The laboratory database 724 can be configured to store laboratory information associated with the plurality of users. The laboratory database 724 can be accessed via a local area network, wide area network, application programming interface, or other suitable communication mechanism. Laboratory information can include results from tests related to blood glucose, complete blood count (CBC), kidney function, liver function, metabolic panel, thyroid test, and urinalysis, among others.

The disease database 726 can be configured to store standardized disease information. The disease database 726 can be accessed via a local area network, wide area network, application programming interface, or other suitable communication mechanism. Standardized disease information can include information related to internal medicine, inherited disease, clinical biochemistry, and pharmacology, as well as a cross-referenced index of human disease, medications, symptoms, signs, and abnormal investigation findings, among others.

The standardized drug database 728 can be configured to store standardized drug information. The standardized drug database 728 can be accessed via a local area network, wide area network, application programming interface, or other suitable communication mechanism. Standardized drug information can include drug product data, drug images, pricing information, professional monographs, patient education and clinical decision support data, among other information.

The research database 730 can be configured to store research information related to a patient, drug, or disease. The research database 730 can be accessed via a local area network, wide area network, application programming interface, or other suitable communication mechanism. Research information can include non-standardized information, information related to trials, FDA approvals, infectious diseases, epidemiology and microbiology.

The EPOR database 708 can include complete clinical and pharmaceutical data for each patient in the form of a patient record 710. Each patient record can have fields or parameters for varying types of data related to a specific patient, including pharmaceutical, clinical, genomic, laboratory, disease, standardized drug, or research information, among other relevant information, populated from the plurality of patient databases 712, 714, 716, 718, the clinical database 720, the genomic database 722, the laboratory database 724, the disease database 726, the standardized drug database 728, and the research database 730, among others. The EPOR can be a consolidated, reconciled database that can be updated periodically, or whenever new data is available for a patient, to help ensure that accurate patient data is available. The fields or parameters of the information related to a particular patient can be reconciled such that differing entries related to an identified patient can be aggregated into a single patient record 710 in the EPOR database 708. By applying predetermined thresholds to the one or more fields of each of the plurality of pharmaceutical databases, a reconciled entry for the identified patient for the entries satisfying the predetermined thresholds. This reconciling has the advantage of optimizing the database performance and storage scheme and preventing a “miss” on critical information related to the identified patient from disparate sources due to differences in name spelling, address, or typos in other user identifiable information, such as social security number, account number, driver's license, or other relevant information.

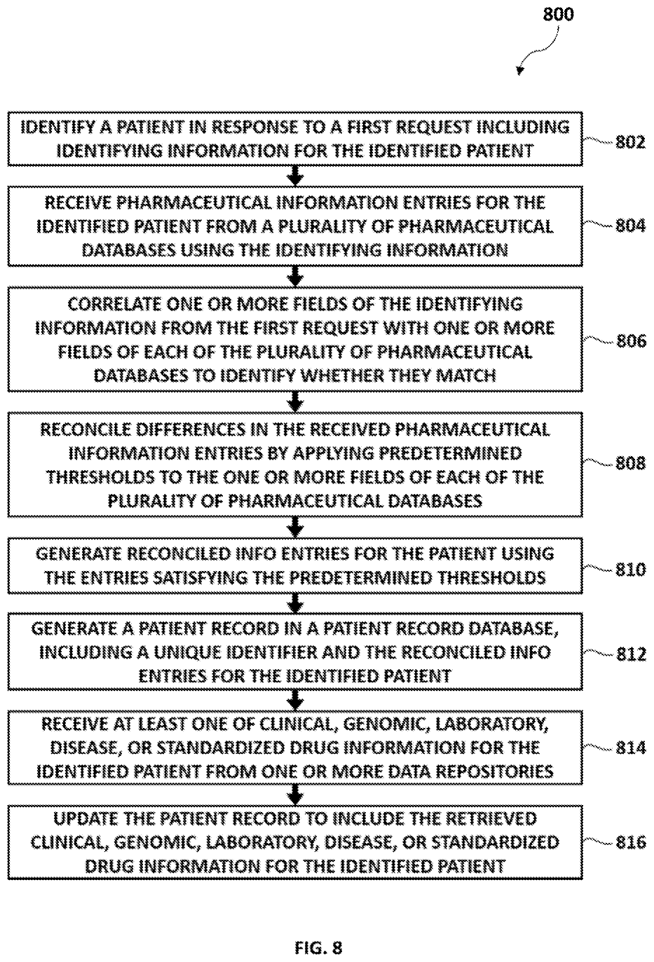

FIG. 8 illustrates a flow chart diagram 800 exemplifying control logic embodying features of a method for generating a portable, interoperable patient medical and pharmaceutical record performed by the system depicted in FIG. 7, in accordance with principles of the present invention. The record generation control logic 800 can be implemented as an algorithm on a server, a machine learning module, or other suitable system. The record generation control logic 800 can be achieved with software, hardware, an application programming interface (API), a network connection, a network transfer protocol, HTML, DHTML, JavaScript, Dojo, Ruby, Rails, other suitable applications, or a suitable combination thereof.

The record generation control logic 800 can leverage the ability of a computer platform to spawn multiple processes and threads by processing data simultaneously. The speed and efficiency of the record generation control logic 800 is greatly improved by instantiating more than one process to generate a record having a patient's data. However, one skilled in the art of programming will appreciate that use of a single processing thread may also be utilized and is within the scope of the present invention.

The record generation control logic 800 process flow of the present embodiment begins at step 802, where the control logic can identify a patient in response to a first request including identifying information for the identified patient. The first request can be received from an EMR, web portal, or other suitable mechanism. The identified patient's identifying information can include a name, birthdate, address, social security number, account number, or other suitable information. The control logic then proceeds to step 804.

At step 804, the record generation control logic 800 can retrieve patient entries from at least one of a plurality of pharmaceutical databases 712, 714, 716, 718, using the identifying information. A database entry for an identified patient can be retrieved by searching a particular pharmaceutical database for fields or parameters matching fields or parameters in the first request. The record generation control logic 800 can access the plurality of pharmaceutical databases 712, 714, 716, 718 via a network connection or other suitable means. The control logic then proceeds to step 806.

At step 806, the record generation control logic 800 can correlate one or more fields of the identifying information from the first request with one or more fields of each of the plurality of pharmaceutical databases 712, 714, 716, 718. For a particular identified patient, the plurality of pharmaceutical databases can have fields or parameters such as name, address, or other user identifiable information, such as social security number, account number, driver's license, or other relevant information. The record generation control logic 800 can correlate the fields or parameters by comparing the two to identify whether they match. The control logic then proceeds to step 808.

At step 808, the record generation control logic 800 can reconcile differences in the retrieved patient entries by applying predetermined thresholds to the one or more fields of each of the plurality of pharmaceutical databases. The one or more fields of each of the plurality of pharmaceutical databases for the identified patient may have variations, such that the fields do not match exactly. For a particular identified patient, the plurality of pharmaceutical databases can have differences in name spelling, address, or typos in other user identifiable information, such as social security number, account number, driver's license, or other relevant information. Entries with these differences may or may not be for the identified patient.

To help reconcile these patient entry differences, predetermined thresholds can be applied to the one or more fields of each of the patient entries, such that a certain tolerance can be attributed to the differences. The predetermined thresholds for the patient entries can be applied to individual fields or the entire patient entry. For example, name misspellings can have a predetermined threshold of a few letters difference between the two entries being reconciled. Additionally, after the predetermined thresholds are applied to the one or more fields of each of the patient entries, predetermined thresholds can be applied to the patient entries in their entirety, such that a certain tolerance can be attributed to the overall differences. For example, total patient entry field differences can have predetermined thresholds such as maximum count or percentage between the two entries being reconciled. If one of the retrieved patient entries meets or exceeds the predetermined thresholds, that patient entry will be excluded from being reconciled with the other patient entries. The control logic then proceeds to step 810.

At step 810, the record generation control logic 800 can generate a reconciled entry for the identified patient with the entries satisfying the predetermined thresholds. The predetermined thresholds can be satisfied by determining whether differences meet, exceed, or fall below the predetermined thresholds, based on the particular application. For example, the record generation control logic 800 can generate a reconciled entry for the identified patient with the entries having a number of differences fall below the predetermined thresholds, or when the entries that match exceed the predetermined thresholds, among other scenarios, based on the particular application. The reconciled entry for the identified patient can include updated information (based on time stamp comparison) for populated fields, additional information (for unpopulated fields), or deletions (for previously populated fields). In this way, the reconciled entry will have the most accurate, up-to-date information regarding the identified patient. The record generation control logic 800 can include a feedback loop such that the thresholds can be modified using other information. For example, the record generation control logic 800 can calculate the differences, standard deviation, average, interpolation, or other data analysis, on the information to set or modify the thresholds. The control logic then proceeds to step 812.

At step 812, the record generation control logic 800 can generate a patient record in a database, including a unique identifier and the reconciled entry for the identified patient. The reconciled entry having the most accurate, up-to-date information regarding the identified patient, can be stored in the EPOR database 708 with the unique identifier. Once a record for a patient has been created, the identifier can be patient identifying information that can be included in a first request. The control logic then proceeds to step 814.

At step 814, the record generation control logic 800 can retrieve at least one of clinical, genomic, laboratory, disease, standardized drug, or research information for the identified patient from one or more data repositories 720, 722, 724, 726, 728, 730. This information can be retrieved for the identified patient by searching one or more data repositories 720, 722, 724, 726, 728, 730 for fields or parameters matching fields or parameters in the first request or other patient-identifying information. The record generation control logic 800 can access the one or more data repositories 720, 722, 724, 726, 728, 730 via a network connection, application programming interface, or other suitable means. The control logic then proceeds to step 816.

At step 816, the record generation control logic 800 can update the patient record to include the retrieved clinical, genomic, laboratory, disease, standardized drug, or research information. The record can add or populate fields for the retrieved clinical, genomic, laboratory, disease, standardized drug, or research information and save the identified patient's updated patient record in the EPOR database 708. The control logic then terminates or awaits a new request identifying a patient and repeats the aforementioned steps.

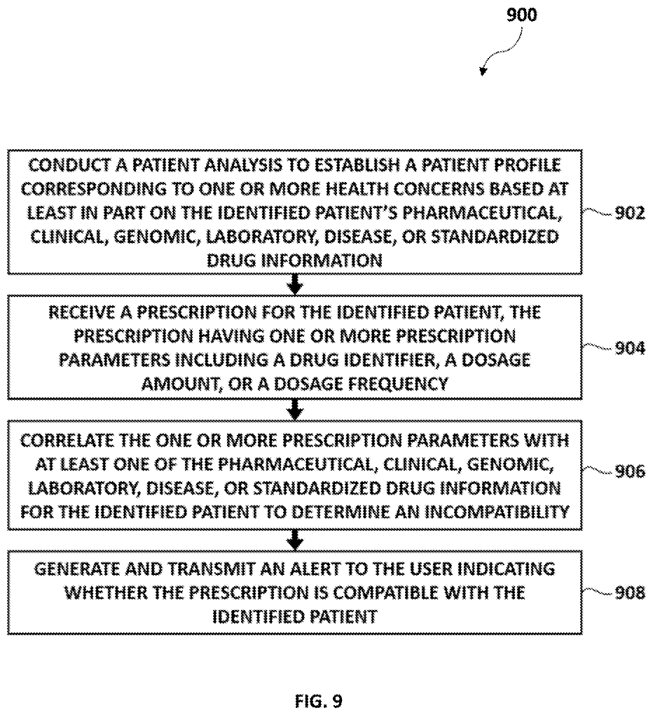

FIG. 9 illustrates a flow chart diagram 900 exemplifying control logic embodying features of a method for determining the compatibility of a prescription for a patient performed by the system depicted in FIG. 7, in accordance with principles of the present invention. The prescription compatibility control logic 900 can be implemented as an algorithm on a server, a machine learning module, or other suitable system. The record generation control logic 800 can be achieved with software, hardware, an application programming interface (API), a network connection, a network transfer protocol, HTML, DHTML, JavaScript, Dojo, Ruby, Rails, other suitable applications, or a suitable combination thereof.

The prescription compatibility control logic 900 process flow of the present embodiment begins at step 902, where the prescription compatibility control logic 900 can conduct a patient analysis to establish a patient profile corresponding to health concerns based at least in part on an identified patient's pharmaceutical, clinical, genomic, laboratory, disease, or standardized drug information. The patient analysis can take into account the identified patient's clinical, genomic, laboratory, disease, standardized drug, or research information. The patient profile can include a listing of health concerns, a health rating, or one of a predetermined number of health concern levels indicating severity of the health concern.

The patient profile can correspond to health concerns based at least in part on the identified patient's pharmaceutical, clinical, genomic, laboratory, disease, or standardized drug information. Such information can include parameters or fields related to specific attributes of the information. A patient's health information or parameters can include patient demographics, individual patient medical information, ethnicity, genomics (including individual genetic data), disease profiles, allergies, allergy profiles, immunizations, blood type, weight, height, pre-existing medical conditions, metabolic rate, current medication usage, blood pressure, red blood cell count, white blood cell count, cholesterol levels, insurance information, drug side effects, and drug cost, among other information or parameters. The control logic then proceeds to step 904.

At step 904, the prescription compatibility control logic 900 can receive a prescription for the identified patient, the prescription having one or more prescription parameters including a drug identifier, a dosage amount, or a dosage frequency. The prescription compatibility control logic 900 can receive a prescription, such as an eScript or other suitable message, for the identified patient from the EMR or web portal. The control logic then proceeds to step 906.

At step 906, the prescription compatibility control logic 900 can correlate the one or more prescription parameters with at least one of the pharmaceutical, clinical, genomic, laboratory, disease, standardized drug, or research information for the identified patient to determine an incompatibility. A patient record can be loaded into memory accessible by the prescription compatibility control logic 900 for further analysis and processing, or a pointer to the record can be utilized by the control logic to further query specific fields of the record in the EPOR database. The prescription compatibility control logic 900 can correlate the fields or parameters by comparing this information with the one or more prescription parameters to identify whether an incompatibility exists based upon the match or non-match of the parameters.

The prescription compatibility control logic 900 can establish thresholds for specific parameters. The thresholds can be binary (present or not present), to indicate whether an incompatibility exists, such as an allergy to a medication (e.g., penicillin, etc.), a food allergy (e.g., dairy, peanut, etc.), whether a prescribed drug would have a negative interaction with a drug the patient is currently taking, among others. The thresholds can also have minimum or maximum values for parameters having a scale, magnitude, or degree, such as a metabolic rate, a body mass index value, a weight, a height, a white blood cell count, among other parameters. The prescription compatibility control logic 900 can include a feedback loop such that the thresholds can be modified using other information. For example, the prescription compatibility control logic 900 can calculate the differences, standard deviation, average, interpolation, or other data analysis, on the information to set or modify the thresholds. The control logic then proceeds to step 908.

At step 908, the prescription compatibility control logic 900 can generate and transmit an alert to the user indicating whether the prescription is compatible with the identified patient. The alert can be transmitted to the EMR, web portal, or other suitable mechanism. The alert can be a CAM, acknowledgement, flag, or other suitable indicator. The control logic then terminates or awaits a new request identifying a patient and repeats the aforementioned steps.

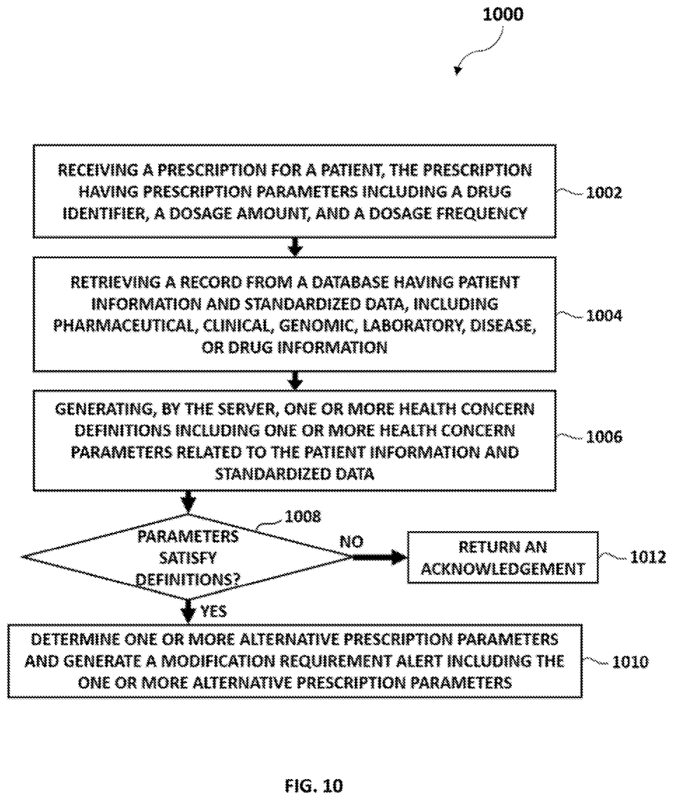

FIG. 10 illustrates a flow chart diagram 1000 exemplifying control logic embodying features of a method for determining prescription modification requirements for a patient performed by the system depicted in FIG. 7, in accordance with principles of the present invention. The prescription modification control logic 1000 can be implemented as an algorithm on a server, a machine learning module, or other suitable system. The prescription modification control logic 1000 can be achieved with software, hardware, an application programming interface (API), a network connection, a network transfer protocol, HTML, DHTML, JavaScript, Dojo, Ruby, Rails, other suitable applications, or a suitable combination thereof.

The prescription modification control logic 1000 process flow of the present embodiment begins at step 1002, where the prescription modification control logic 1000 can receive a prescription for a patient, the prescription having prescription parameters including a drug identifier, a dosage amount, and a dosage frequency. The prescription modification control logic 1000 can receive a prescription, such as an eScript or other suitable message, for the identified patient from the EMR or web portal. The prescription can have one or more parameters or fields describing characteristics of the prescription. The prescription can also include patient-identifying information, such as personal information or a unique identifier. The control logic then proceeds to step 1004.

At step 1004, the prescription modification control logic 1000 can import a record from a database having patient information and standardized drug data, including pharmaceutical, clinical, genomic, laboratory, disease, or drug information. The prescription modification control logic 1000 can use the patient identifying information to query the EPOR database to identify the identified patient's record. The patient record can be loaded into memory accessible by the prescription modification control logic 1000 for further analysis and processing, or a pointer to the record can be utilized by the control logic to further query specific fields of the record in the EPOR database. The control logic then proceeds to step 1006.

At step 1006, the prescription modification control logic 1000 can generate one or more health concern definitions including one or more health concern parameters related to the patient information and standardized drug data. A health concern definition can establish rules and thresholds for parameters. The parameters can be fields in a record or entry, a CAM, or other suitable information element. The prescription modification control logic 1000 can include a feedback loop such that the thresholds can be modified using other information. For example, the prescription modification control logic 1000 can calculate the differences, standard deviation, average, interpolation, or other data analysis, on the parameters or information to set or modify the thresholds or health concern definitions. The thresholds can be binary (present or not present), to indicate whether an incompatibility exists, such as an allergy to a medication (e.g., penicillin, etc.), a food allergy (e.g., dairy, peanut, etc.), whether a prescribed drug would have a negative interaction with a drug the patient is currently taking, among others. The thresholds can also have minimum or maximum values for parameters having a scale, magnitude, or degree, such as a metabolic rate, a body mass index value, a weight, a height, a white blood cell count, among other parameters. The control logic then proceeds to step 1008.

At step 1008, the prescription modification control logic 1000 determines, responsive to the server, whether one or more of the prescription parameters satisfy one or more parameters of the health concern definitions. The health concern definitions can be satisfied by determining whether differences meet, exceed, or fall below the thresholds or health concern definitions, based on the particular application. For example, the prescription modification control logic 1000 can correlate one or more of the prescription parameters with one or more parameters of the health concern definitions for the identified patient to determine any differences and whether those differences fall below the predetermined thresholds, match the predetermined thresholds, or exceed the predetermined thresholds, among other suitable definitions. The health concern definitions relate to health concerns or potential issues for the identified patient related to health related information. If the prescription parameters satisfy one or more parameters of the health concern definitions, the control logic proceeds to step 1010. If the prescription parameters do not satisfy one or more parameters of the health concern definitions, the control logic proceeds to step 1012.

At step 1010, the prescription modification control logic 1000 can determine one or more alternative prescription parameters and return a modification requirement alert including the one or more alternative prescription parameters.

For example, the identified patient may have a weight that falls below a threshold for a dosage amount of the prescription parameter, based on information retrieved from the standardized drug database 728. The patient's weight may have not been considered by the prescribing doctor when the prescription was first written, or the patient's weight may have changed during a subsequent office visit before the prescription was elected to be filled. The prescription modification control logic 1000 can generate an alternative prescription parameter regarding the dosage amount, using the information retrieved from the standardized drug database 728. A modification requirement alert can be returned to the user along with the alternative prescription parameter regarding the dosage amount.

In another example, the identified patient may be taking a medication that he or she failed to disclose to the prescribing physician. The physician may have then prescribed a prescription for a drug having a particular drug identifier. The prescription modification control logic 1000 can query the EPOR database to determine whether any prescription conflicts exist by checking the patient's prescription history. If the drug identifier prescription parameter satisfies one or more parameters of the health concern definitions (such as meeting the incompatibility), based on drug incompatibility information retrieved from the standardized drug database 728, a binary indication of the presence of the drug incompatibility can generate a modification requirement alert. The prescription modification control logic 1000 can retrieve the patient's condition information from the clinical database 720 and query the standardized drug database 728 to find similar drugs that can treat the same condition, but that do not have an incompatibility. The prescription modification control logic 1000 can then generate an alternative prescription parameter regarding the drug identifier, using the information retrieved from the standardized drug database 728 and the clinical database 720. The modification requirement alert can be returned to the user along with the alternative prescription parameter regarding the dosage amount.

Many additional examples exist. Any suitable combination of information and parameters discussed in the present disclosure can be utilized to identify health concerns and generate alerts. The prescription modification control logic 1000 can generate a modification requirement alert via a CAM, display pop-up, e-mail, text message, or other suitable means of communication. The control logic then terminates or awaits a new request identifying a patient and repeats the aforementioned steps.

At step 1012, the prescription modification control logic 1000 can return an acknowledgement. If no health concerns are detected by the prescription modification control logic 1000, an acknowledgment can be generated by the control logic 1000 to indicate that the prescription may be filled. The acknowledgement can be returned to the user via the EMR or web portal, or provide the eScript or other suitable message directly to the fulfilment entity for fulfilment. The prescription modification control logic 1000 can generate an acknowledgement and return the acknowledgment to the user via a CAM, display pop-up, e-mail, text message, or other suitable means of communication. The control logic then terminates or awaits a new request identifying a patient and repeats the aforementioned steps.

### Technological Summary

The present invention provides a new system architecture for processing healthcare data that is depicted in FIGS. 1, 2, 3, and 7. The invention comprises a combination of data processing mechanisms arranged and configured for efficiently processing both prescription submissions and fulfillment, as well as healthcare data updates and safe, secure patient outcome data accessibility in real time to all authorized healthcare providers and patients. Certain embodiments also include intelligent thresholding processes to ensure identification of healthcare concerns.

The technological improvements embodied in the present invention include configuring the following three innovations: (1) an enhanced database organization system; (2) an enhanced (automated) prescription submission and fulfillment processing system provided by a new clinical services platform; (3) a data file format for facilitating data movement and processing in both systems (1) and (2); (4) an improved computer platform for generating a portable, interoperable patient medical and pharmaceutical record; (5) an improved computer platform having a machine learning module for determining the compatibility of a prescription for a patient; and (6) an improved computer platform having a machine learning module for determining prescription modification requirements for a patient.

The submission and fulfillment processing system draws its data from two principal databases, the ePR (pharmaceutical data) and EPO data (standardized healthcare data) databases, both of which contain volumes of data formatted in disparate configurations from multiple sources.

Part of the job of populating the third principle database—the consolidated EPOR database containing a complete patient healthcare outcome record—includes translating the disparate formats of the source data into a common format (such as XML, for example) for the EPOR database. The clinical services platform contains the mechanisms for (A) translating these data, (B) installing them in an organized way in the EPOR database; (C) maintaining the record in the EPOR updated with every prescription processing and other healthcare provider transaction; and (D) enabling real time, interoperable access available to all authorized providers and patients of data updated with every healthcare transaction. An important ingredient of the processing system that facilitates the movement of data in the functions of the clinical services platform is the clinical analytical message (“CAM”) data file.

Accordingly, a novel patient-centric, portable, interoperable, interdisciplinary, electronic patient outcome record and system, accessible in real time by authorized healthcare providers and their patients through a website portal into the system, is provided by the present invention. The system comprises the novel combination of a single healthcare database, a clinical services platform, and a clinical analytical message data file. Each of these three innovations in the technology of healthcare data processing are specifically configured for their respective data processing functions. The advantage of this approach is that the system could not otherwise be configured to perform its functions in real time because the compromises in security, and speed and resulting bottlenecks associated with modified traditional structures are eliminated. The efficiencies resulting from the dedicated architecture and speed—and the absence of bottlenecks—engineered into the system are essential to provide a system capable of responding to the extraordinary growth of healthcare data needs now and in the foreseeable future. It is this combination, and the unique characteristics of each of these elements working together that provides the secure, real time access and processing of prescription drug submission and fulfillment transactions associated with treatment of a patient by a healthcare provider. Moreover, the system as thus constructed provides enhanced patient safety and security of healthcare information while providing up-to-date patient outcome data in compliance with federal regulations.

The consolidated healthcare database is populated with pharmaceutical and standardized healthcare data at every prescription transaction, including translation of the native data format of the source data into a common, readily accessible data format. The clinical services platform, through its interactions with the active components of the system, using the suite of mechanisms both structural and algorithmic directs the processing for both constructing and maintaining the single healthcare database and for processing the submission and fulfillment of prescriptions submitted by the patient's healthcare providers. The data transactions and processes carried out by the clinical services platform with its review processor, operating in conjunction with the single healthcare database, are enabled and facilitated in real time by a unique clinical analytical message data file that conveys both data and process commands within the system and with external entities such as a transaction hub (the “switch”), claim processing entities, payers, and drug manufacturers.

Persons skilled in the art will readily understand that these advantages and objectives of this system that provides real time, interoperable updates and access to a patient's secure healthcare data would not be possible without the particular combination of computer hardware and other structural components and mechanisms assembled in this inventive system and described herein. It will be further understood that a variety of programming tools, known to persons skilled in the art, are available for implementing the control of the features and operations described in the foregoing material. Moreover, the particular choice of programming tool(s) may be governed by the specific objectives and constraints placed on the implementation plan selected for realizing the concepts set forth herein and in the appended claims.

The invention may be embodied in other specific forms without departing from the spirit or essential characteristics thereof. For example, each of the new structures described herein, such as the EPOR database, the clinical services platform, the review processor, and the CAM data file may be modified to suit particular local variations or requirements while retaining their basic configurations or structural relationships with each other or while performing the same or similar functions described herein. The present embodiments are therefore to be considered in all respects as illustrative and not restrictive. Accordingly, the scope of the invention is established by the appended claims rather than by the foregoing description. All changes which come within the meaning and range of equivalency of the claims are therefore intended to be embraced therein. Further, the individual elements of the claims are not well-understood, routine, or conventional. Instead, the claims are directed to the unconventional inventive concept described in the specification.

In another example, a potential use for the CAM data file may be during the submission review process to determine whether the submitted prescription is for a specialty drug that requires delivery of specific clinical and educational information to the patient or is not for a specialty drug, which ordinarily does not require such specific information be delivered to the patient. A feature of the CAM data file in that instance may be to identify drugs with certain associated risks that suggest the patient be counseled by the pharmacy to identify patient-specific factors or vulnerabilities to those risks.

What is claimed is:

1. A system for determining the compatibility of a prescription for a patient, the system including a server having a patient record database, and a machine learning module having a processor, the system comprising:
a server including computer-executable instructions that when executed cause the server to:
identify a patient, in response to a first request from a user via a first computing device over an encrypted network, the first request including identifying information for the identified patient,
receive pharmaceutical information entries for the identified patient from a plurality of pharmaceutical databases using the identifying information,
reconcile, via a machine learning module, differences in the received pharmaceutical information entries by applying predetermined thresholds to the one or more fields of each of the plurality of pharmaceutical databases and generating reconciled pharmaceutical information entries for the identified patient using the pharmaceutical information entries satisfying the predetermined thresholds,
generate a patient record in a patient record database, including a unique identifier and the reconciled pharmaceutical information entries for the identified patient,
receive at least one of clinical, genomic, laboratory, disease, or standardized drug information for the identified patient from one or more data repositories, and
update the patient record to include the received clinical, genomic, laboratory, disease, or standardized drug information for the identified patient; and
a machine learning module having a processor configured to:
conduct a patient analysis to establish a patient profile corresponding to one or more health concerns based at least in part on the identified patient's pharmaceutical, clinical, genomic, laboratory, disease, or standardized drug information,
receive a prescription for the identified patient, the prescription having one or more prescription parameters including a drug identifier, a dosage amount, or a dosage frequency,
correlate the one or more prescription parameters with at least one of the pharmaceutical, clinical, genomic, laboratory, disease, or standardized drug information for the identified patient to determine an incompatibility, and
generate and transmit an alert to the user indicating whether the prescription is compatible with the identified patient.

2. The system of claim 1, further comprising correlating, via the server, one or more fields of the identifying information from the first request with one or more fields of each of the plurality of pharmaceutical databases to identify whether they match.

3. The system of claim 1, wherein the identified patient's pharmaceutical, clinical, genomic, laboratory, disease, or standardized drug information includes parameters or fields related to specific attributes of the patient's information.

4. The system of claim 1, wherein the patient profile includes a listing of health concerns, a health rating, or one of a predetermined number of health concern levels indicating severity of the health concern.

5. The system of claim 1, wherein the thresholds have minimum or maximum values for parameters having a scale, magnitude, or degree.

6. The system of claim 1, wherein the system further comprises a feedback loop configured to modify the thresholds using the patient record.

7. The system of claim 6, wherein the machine learning module modifies the thresholds by calculating a difference, standard deviation, average, or interpolation of selected fields of the patient record.

8. The system of claim 1, wherein the patient analysis to establish the patient profile generates a health concern level for a patient.

9. The system of claim 1, wherein the machine learning module generate one or more health concern definitions to establish the thresholds for the prescription parameters.

10. The system of claim 1, wherein the alert is a clinical analytical message (CAM) data file.

11. A computer-implemented method for determining the compatibility of a prescription for a patient, the computer-implemented method comprising:
identifying, via a server, a patient, in response to a first request from a user via a first computing device over an encrypted network, the first request including identifying information for the identified patient;
receiving, via the server, pharmaceutical information entries for the identified patient from a plurality of pharmaceutical databases using the identifying information;
reconciling, via a machine learning module, differences in the received pharmaceutical information entries by applying predetermined thresholds to one or more fields of each of the plurality of pharmaceutical databases and generating reconciled pharmaceutical information entries for the identified patient using the pharmaceutical information entries satisfying the predetermined thresholds;
generating, via the server, a patient record in a patient record database, including a unique identifier and the reconciled entries for the identified patient;
receiving, via the server, at least one of clinical, genomic, laboratory, disease, or standardized drug information for the identified patient from one or more data repositories; and
updating, via the server, the patient record to include the received clinical, genomic, laboratory, disease, or standardized drug information for the identified patient;
conducting, via a machine learning module having a processor, a patient analysis to establish a patient profile corresponding to health concerns based at least in part on the identified patient's pharmaceutical, clinical, genomic, laboratory, disease, or standardized drug information;
receiving, via the server, a prescription for the identified patient, the prescription having one or more prescription parameters including a drug identifier, a dosage amount, or a dosage frequency;
correlating, via the machine learning module having a processor, the one or more prescription parameters with at least one of the pharmaceutical, clinical, genomic, laboratory, disease, or standardized drug information for the identified patient to determine an incompatibility; and
generating, via the server, an alert indicating whether the prescription is compatible with the identified patient and transmitting the alert to the computing device over the encrypted network.

12. The computer-implemented method of claim 11, further comprising correlating, via the server, one or more fields of the identifying information from the first request with one or more fields of each of the plurality of pharmaceutical databases to identify whether they match.

13. The computer-implemented method of claim 11, wherein the identified patient's pharmaceutical, clinical, genomic, laboratory, disease, or standardized drug information includes parameters or fields related to specific attributes of the patient's information.

14. The computer-implemented method of claim 11, wherein the patient profile includes a listing of health concerns, a health rating, or one of a predetermined number of health concern levels indicating severity of the health concern.

15. The computer-implemented method of claim 11, wherein the thresholds have minimum or maximum values for parameters having a scale, magnitude, or degree.

16. The computer-implemented method of claim 11, wherein the system further comprises a feedback loop configured to modify the thresholds using the patient record.

17. The computer-implemented method of claim 16, wherein the machine learning module modifies the thresholds by calculating a difference, standard deviation, average, or interpolation of the patient record.

18. The computer-implemented method of claim 11, wherein the patient analysis to establish the patient profile generates a health concern level for a patient.

19. The computer-implemented method of claim 11, wherein the machine learning module generate one or more health concern definitions to establish the thresholds for the prescription parameters.

20. The system of claim 11, wherein the alert is a clinical analytical message (CAM) data file.
